(19) United States
(12) Patent Application Publication
(10) Pub. No.: US 2023/0233750 A1
(43) Pub. Date: Jul. 27, 2023

(54) ENDOTRACHEAL TUBE

(71) Applicant: SWIFTSURE INNOVATIONS INC., Pasadena (CA)

(72) Inventors: Deanne McCarthy, Pasadena (CA); Bradley Morris, Corner Brook (CA); Kathryn Atwell, Halifax (CA); Joel Ironstone, Toronto (CA); Matthew Asselin, Toronto (CA); Christopher Gillespie, Toronto (CA)

(21) Appl. No.: 18/011,421

(22) PCT Filed: Jun. 18, 2021
(86) PCT No.: PCT/CA2021/050835

§ 371 Date: Dec. 19, 2022

Related U.S. Application Data
(60) Provisional application No. 63/201,398, filed on Apr. 28, 2021.
(60) Provisional application No. 63/199,994, filed on Feb. 8, 2021.
(60) Provisional application No. 63/198,641, filed on Oct. 30, 2020.
(60) Provisional application No. 62/706,769, filed on Sep. 9, 2020.
(60) Provisional application No. 63/041,365, filed on Jun. 19, 2020.

(51) Int. Cl. A61M 3/02; A61M 16/04; A61M 1/00

(52) U.S. Cl. CPC A61M 3/027; A61M 16/0463; A61M 1/774; A61M 3/0283; A61M 1/772; A61M 16/049; A61M 2205/0205; A61M 2205/0222; A61M 2202/0468; A61M 2210/0625; A61M 2210/0637

(57) ABSTRACT

A device for irrigation of an oral cavity in a patient includes a suction element configured to be disposed in the oral cavity and configured to suction a fluid out of the oral cavity. The suction element also has one or more irrigation outlets configured to irrigate the oral cavity with the fluid. A suction line is fluidly coupled with the suction element and is configured to be fluidly coupled with a vacuum source. An irrigation line is fluidly coupled with the one or more irrigation outlets and is fluidly coupled with a source of the fluid.

### CROSS-REFERENCE TO RELATED PATENT DOCUMENTS

The present application is a non-provisional of, and claims the benefit of US Provisional Patent Application Nos. 63/041,365 (Attorney Docket No. 5619.001PRV) filed on Jun. 19, 2020; 62/706,769 (Attorney Docket No. 5619.001PV2) filed on Sep. 9, 2020; 63/198,641 (Attorney Docket No. 5619.001PV3) filed on Oct. 30, 2020; 63/199,994 (Attorney Docket No. 5619.001PV4) filed on Feb. 8, 2021; and 63/201,398 (Attorney Docket No. 5619.001PV5) filed on Apr. 28, 2021; the entire contents of each are incorporated herein by reference.

### BACKGROUND

Mechanically ventilated patients require a tube to be inserted into the trachea so that a ventilator can deliver oxygen to the lungs. The tube may be an endotracheal tube, or a tube introduced via a tracheostomy. The proximal end of the tube is coupled to tubing that is attached to a ventilator so that air can be delivered to and from the lungs and the distal end of the tube is disposed below the glottis where the trachea branches into the bronchial tubes. A cuff on the tube expands into engagement with the tracheal wall forming a seal which permits ventilation and also prevents fluids and other material from being aspirated into the lungs.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows basic anatomy of a patient's mouth, trachea, and esophagus.

FIG. 2 illustrates an example of an endotracheal tube.

FIG. 3 shows a top view of a mouth pad.

FIG. 4 shows a side view of a mouth pad coupled to an endotracheal tube.

FIGS. 4A-4B show another example of a mouth pad.

FIG. 4C shows still another example of a mouth pad.

FIG. 4D shows an example of a mouth pad assembly.

FIGS. 4E and 4F show a side view and a top view, respectively, of another example of a mouth pad.

FIG. 4G shows an example of an anchor element on a mouth pad.

FIG. 4H shows an example of a mouth guard on a mouth pad.

FIGS. 4I-4L shows examples of fastener elements on a mouth pad.

FIG. 4M show an example of a headband used with a mouth pad.

FIG. 5 shows an optional cross-section of the endotracheal tube in FIG. 2.

FIG. 6 shows an optional cross-section of the endotracheal tube in FIG. 2.

FIGS. 7A-7U illustrate an example of intubation using the endotracheal tube of FIG. 2.

FIGS. 8A-8C illustrate an example of a mouth pad with a fastener.

FIGS. 9A-9B illustrate another example of a mouth pad with a fastener.

FIGS. 10A-10B illustrate still another example of a mouth pad with a fastener.

FIGS. 11A-11B show an example of a mouth pad with two discrete portions.

FIG. 12 shows an example of an endotracheal tube with various lines coupled to the endotracheal tube.

FIG. 13 shows an example of an endotracheal tube with various lines coupled to the endotracheal tube and a mouth pad.

FIGS. 14A-14C show another example of a mouth pad.

FIGS. 15A-15B show perspective views of an irrigation and suction device for an oral cavity.

FIG. 16A shows a partial top perspective view of the device in FIGS. 15A-15B.

FIG. 16B shows a partial bottom perspective view of the device in FIGS. 15A-15B.

FIG. 16C shows a partial bottom perspective view of the device in FIGS. 15A-15B.

FIGS. 17A-17B show examples of a suction guard on an irrigation device.

FIG. 18 shows an irrigation device coupled to an endotracheal tube.

FIG. 19 shows use of the irrigation device in FIGS. 15A-15B in a patient.

FIG. 20 shows a cross-section of a suction element disposed in an oral cavity.

FIG. 21 shows a patient laying on a lateral side.

### DETAILED DESCRIPTION

Mechanically ventilated patients require a tube to be inserted into the trachea so that a ventilator can deliver oxygen to the lungs. The tube may be an endotracheal tube, or a tube introduced via a tracheostomy. The proximal end of the tube is coupled to tubing attached to a ventilator so that air can be delivered to and from the lungs and the distal end of the tube is disposed below the glottis where the trachea branches into the bronchial tubes. A cuff on the tube expands into engagement with the tracheal wall forming a seal which permits air from passing the cuff so that ventilation may be properly performed, and the cuff also helps to prevent fluids and other material from being aspirated into the lungs.

However, secretions or other fluid containing pathogens may collect in the subglottic space and these secretions and fluids may still cross the seal formed by the cuff and flow into the distal trachea and bronchopulmonary airway causing ventilator acquired pneumonia (VAP).

VAP has the highest mortality rate of any hospital acquired infection. VAP is a nosocomial (hospital acquired) pneumonia occurring in a patient after 48 hours of mechanical ventilation and it is the most common infectious complication in critically ill patients. In the USA, between 9% and 25% of patients in intensive care units will acquire pneumonia via mechanical ventilators while hospitalized with a mortality rate of 25%. The increased cost associated from VAP is estimated to be between $40,000 and $60,000 USD per patient.

Exogenous and endogenous mechanisms predispose a patient to VAP. Exogenous mechanisms include bacterial contamination of ventilators, air, or the environment which can be introduced into the tracheobronchial tree. Endogenous mechanisms of VAP may result from pathogens that colonize in cavities that create a reservoir for pathogens, such as the pharynx and craniofacial cavities. Clinical research suggests that endogenous mechanisms may be the most significant cause of VAP, and the oral and nasal cavity may be the most significant source of bacteria that causes infection in the lungs. Biofilm formation likely begins in the mouth and then progresses to the endotracheal tube itself. The advanced biofilm stage (mature stage) may be linked with bacterial shedding, aspiration of the pathogens and subsequent pneumonia. Examples of the device disclosed herein may be used to routinely cleanse the areas that serve as a reservoir for pathogens which may prevent or dramatically slow the formation of biofilms that contain pathogenic microorganisms.

Additionally, it may be advantageous to provide a device and method of use which may be helpful in preventing VAP by isolating the oral and nasal cavities from the lower respiratory tract for the purpose of irrigation in a mechanically ventilated patient without risk of aspiration into the lungs. The device may remove bacteria and organic debris from the nasal and oral cavities, the two main reservoirs for endogenous pathogens that promote proliferation of VAP. Such a device may allow for irrigation and suctioning of irrigation fluid without the required use of external suction catheters and therefore decreases risk of mucous membrane injury. The device may also allow for irrigation without dwell time, meaning that solution can be introduced and simultaneously suctioned out. Such devices and methods may be cost effective, efficient, and easy to use, allowing for frequent intervention.

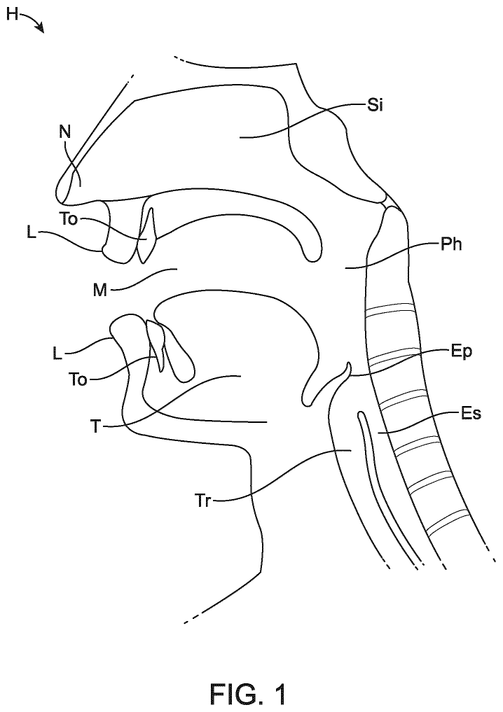

FIG. 1 shows basic human anatomy of a patient's mouth and throat area. The patient's head H includes lips L, teeth To, mouth M, a tongue T, nose N, sinus cavity Si, pharynx Ph which includes the nasopharynx, oropharynx and hypopharynx regions, epiglottis Ep, esophagus Es and trachea Tr. The pharynx is the region of the throat where the rear of the mouth and nasal cavity converges, and it is above the esophagus and larynx. The pharynx region includes an upper portion referred to as the nasopharynx, a lower portion referred to as the hypopharynx, and the portion therebetween is referred to as the oropharynx. The larynx turns into the trachea Tr which then transitions into the bronchial passageways. The esophagus is coupled with the stomach, and the epiglottis is a valve that prevents fluids, food, or other unwanted substances from entering the trachea and aspirating into the lungs. The nares in the nose N open into the sinus cavity Si which then connects to the back of mouth at the pharynx.

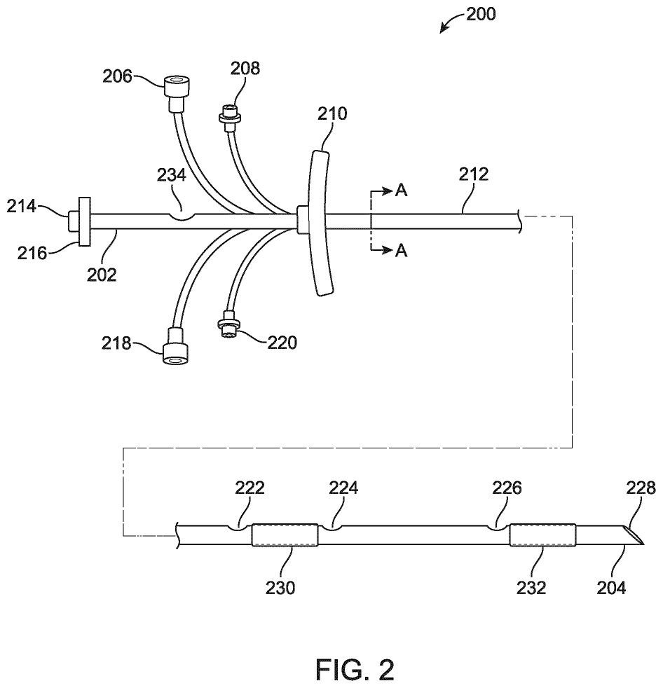

FIG. 2 shows an example of an endotracheal tube that may be used to intubate a patient and may help reduce ventilator acquired pneumonia (VAP). The endotracheal tube includes a first lower expandable cuff to seal the tube in the trachea and also includes a second expandable cuff that is placed anywhere above the vocal chords in the larynx while still allowing irrigation fluid introduced into the nares to be suctioned out through the suction lumen within the endotracheal tube above the second cuff. Therefore, the second cuff must not be placed so high in the patient as to occlude the nasopharynx and prevent irrigation fluid introduced into the nares from being suctioned out by a port superior to the upper cuff. This region will be referred to hereinafter as the region above the vocal chords in the larynx. This region includes the larynx as well as the pharynx which comprises the nasopharynx, oropharynx, and hypopharynx, as well as areas superior and inferior thereto. The second cuff when expanded forms a seal which allows large volume irrigation/lavage which can reduce bacterial and viral biofilm in the mouth and nares and therefore may minimize bacterial migration past the endotracheal tube. A fenestration in the endotracheal tube allows suctioning of pooled and contaminated secretions proximal to the subglottic cuff (lower cuff) which otherwise may enter the tracheal-bronchial tree. Alleviating or minimizing microorganisms with the endotracheal tube and associated method of use may help alleviate VAP in patients.

The endotracheal tube 200 includes an elongate shaft 212 having a proximal end 202 and a distal end 204. The elongate tube may be any length or size to accommodate differing patient anatomies. A lumen extends between the proximal and distal ends 202, 204. Aperture 214 on the proximal end and aperture 228 on the distal end allow fluid such as a gas like air or oxygen, or a liquid to enter the endotracheal tube via the proximal aperture 214, pass through the lumen and exit the distal aperture 228. In the case of ventilation, air would be delivered to the lungs.

The proximal end 202 of the elongate tube may include a connector 216 which allows the endotracheal tube to be releasably coupled with tubing that is attached to a ventilator. The elongate tube 212 includes an upper expandable cuff 230 (sometimes also referred to herein as the pharyngeal or hypopharyngeal cuff, or second/upper cuff above the vocal chords) and a lower expandable cuff 232 (sometimes also referred to herein as the subglottic or tracheal cuff). The upper expandable cuff may be a balloon or other expandable member that may be disposed in the patient's pharynx which includes the nasopharynx, oropharynx, hypopharynx or anywhere above the vocal chords in the larynx, to form a seal between the tissue in that area and the elongate shaft 212. Similarly, the lower expandable cuff 232 may be a balloon or other expandable member that may be disposed in the glottic or subglottic region of the patient to form a seal between tissue in the trachea and the elongate shaft. Each expandable cuff is fluidly coupled with a discrete lumen extending through a wall of elongate shaft and extending proximally to a separate port for each lumen/expandable cuff as will be illustrated later. Either expandable cuff 230, 232 may be a cylindrical balloon, an annulus, a toroidal shape, a spherical shape, or any other shaped disposed circumferentially around the elongate shaft so that when inflated, the cuff will form a seal between the endotracheal tube and adjacent anatomy. Other optional shapes are disclosed below.

Fenestrations 222 and 226 are apertures that extend through the wall of the elongate shaft 212. The upper fenestration 222 is superior to the upper expandable cuff 230, and the lower fenestration 226 is superior to the lower expandable cuff 232. Each fenestration is fluidly coupled with its own discrete lumen in the wall of the elongate shaft 212 and that lumen extends proximally from the fenestration toward the proximal end of the elongate shaft and is fluidly coupled with a port on the proximal end of the endotracheal tube as will be explained. Thus, fluid such as a liquid or a gas may be introduced into a port at the proximal end of the endotracheal tube and delivered via a lumen to a fenestration near the distal end of the endotracheal tube and then introduced from the fenestration to the adjacent anatomy.

Optional fenestrations 224 and 234 are fluidly coupled together via a separate and discrete lumen extending through the wall of the elongate shaft. Lower fenestration 224 is disposed inferior to the upper expandable cuff, and the upper fenestration 234 is disposed closer to the proximal end 202 of the elongate shaft. Fenestrations 224, 234 optionally allow a feeding tube such as an orogastric feeding tube to be introduced through fenestration 234, advanced distally through its own discrete lumen and exit fenestration 224 where the tube may be further advanced into the esophagus and stomach.

Any of the fenestrations 234, 222, 224, 226 may have any shape including round, square, rectangular, elliptical, oval, or any other desired shape, and they may have any desired size.

FIG. 2 shows four ports 206, 208, 218, and 220 coupled to the proximal end of the elongate shaft 212. Each port may include a connector such as Luer connector, barbed fitting, stopcock, or any other connector that is fluidly coupled with a short length of tubing. The tubing is fluidly coupled to a discrete lumen that is then fluidly coupled with an expandable cuff or fenestration. For example, here port 206 is fluidly coupled with a lumen that is fluidly coupled with fenestration 222 and port 208 is fluidly coupled with a lumen that is fluidly coupled with expandable cuff 230. Port 218 is fluidly coupled with a lumen that is fluidly coupled with fenestration 226 and port 220 is fluidly coupled with a lumen that is fluidly coupled with expandable cuff 232. Therefore, fluid input in port 206 exits fenestration 222 and fluid input in port 208 expands cuff 230. Similarly, fluid removed from fenestration 222 may be removed via port 206, and fluid removed from expandable cuff 230 may be removed via port 208 to deflate cuff 230. Fluid introduced into port 218 exits fenestration 226 and fluid introduced into port 220 exits into and fills expandable cuff 232. Fluid removed from fenestration 226 may be removed from port 218 and fluid removed from expandable cuff 232 may be removed via port 220 to deflate cuff 232. Fluid may be introduced or removed from the ports with a syringe or any other device. FIG. 2 shows the ports at approximately the same axial position along the longitudinal axis of the elongate shaft. However, the ports may be axially offset from one another to facilitate easy identification by the operator. For example ports 218 and 220 may be disposed more distally relative to ports 206 and 208 so the user knows that the more distal ports 218, 220 correspond with the more distal cuff 232 and the more distal fenestration 226 while the more proximal ports 206, 208 correspond with the more proximal fenestration 222 and more proximal cuff 230, respectively.

An optional mouth pad 210 may be slidably disposed on elongate shaft 212 and it may be disposed distal of the ports 206, 208, 218, 220 so that it may be advanced over the elongate shaft toward the patient's mouth without binding on the tubing in ports 206, 208, 218, 220. Additional disclosure on the mouth pad is discussed herein along with other mouth pads (e.g. mouth pad 400) which may also be used instead of mouth pad 210.

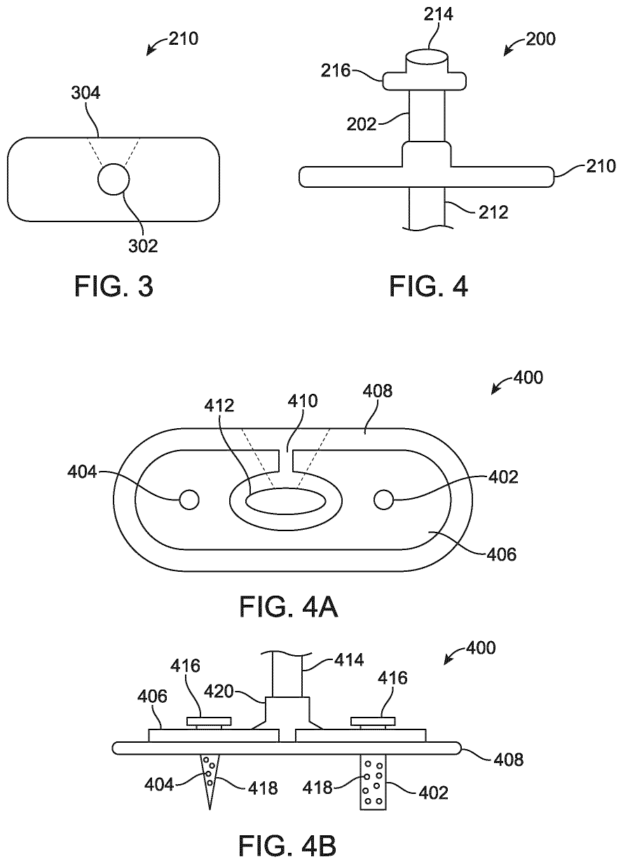

FIG. 3 shows a side view of mouth pad 210 which may be the same mouth pad shown in FIG. 2. The mouth pad 210 may be any shape but in this example is a thin rectangular and planar pad with a central hole 302 extending through the pad and a slit region 304 also extending through the thickness of the pad and connecting an outer edge of the pad with the central hole 302. The corners of the pad may be rounded to avoid forming any sharp edges. The central hole 302 is sized to receive the elongate shaft of the endotracheal tube as seen in FIG. 2. The slit region 304 allows an operator to slide the mouth pad over the elongate shaft so that the elongate tube is disposed in the central hole 302. The mouth pad may then be slidably disposed along the elongate shaft. The mouth pad may be formed of any number of materials such as soft resilient materials like silicone, polyurethane, or other materials known in the art. It may be formed with a clear material to allow observation into the mouth or it may be opaque. The mouth pad in use may be slidably advanced along the elongate tube into apposition with the lips on the patient's mouth to close the mouth and form a temporary seal as will be discussed further below.

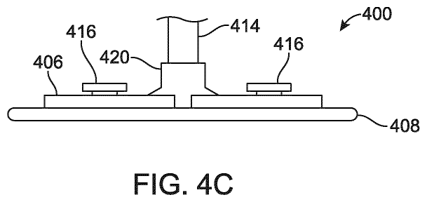

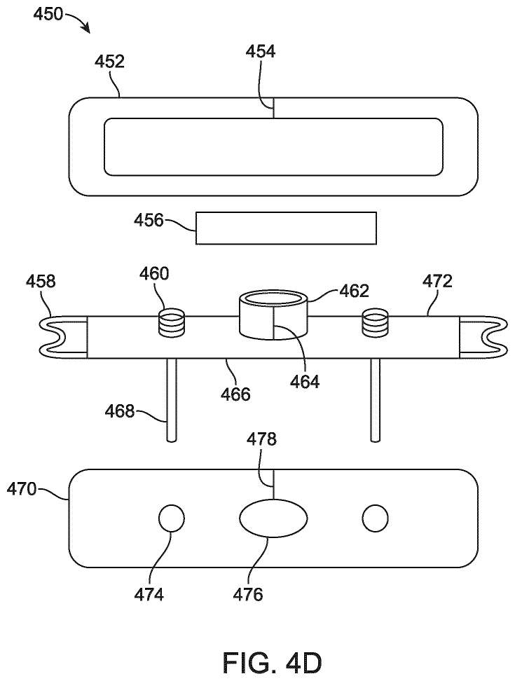

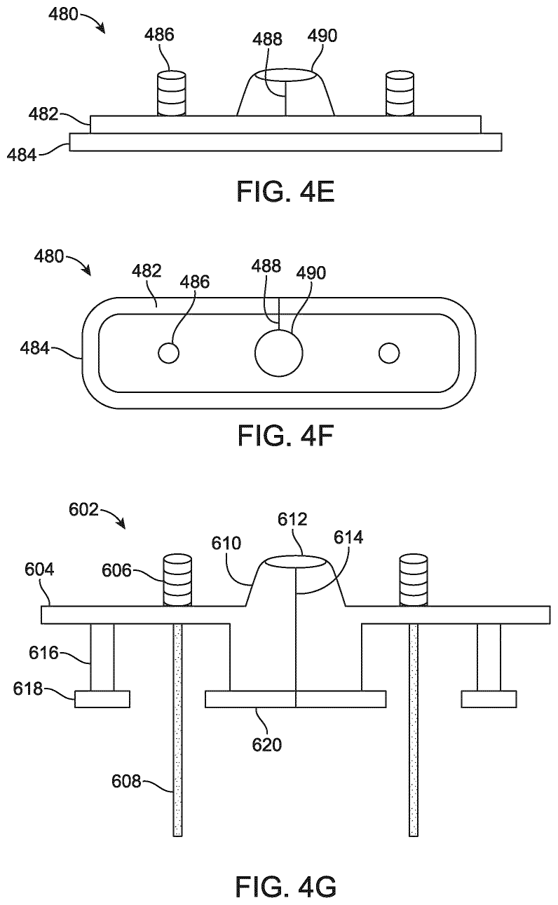

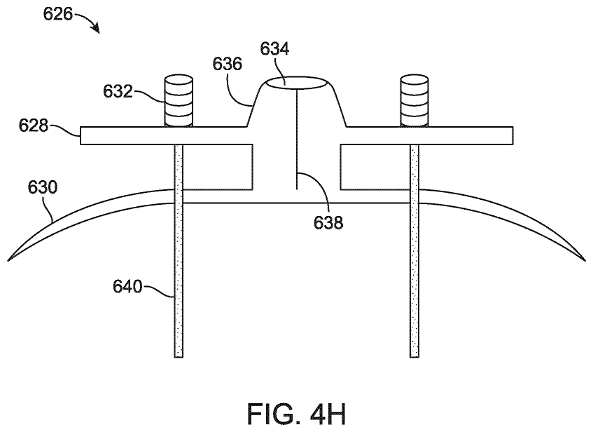

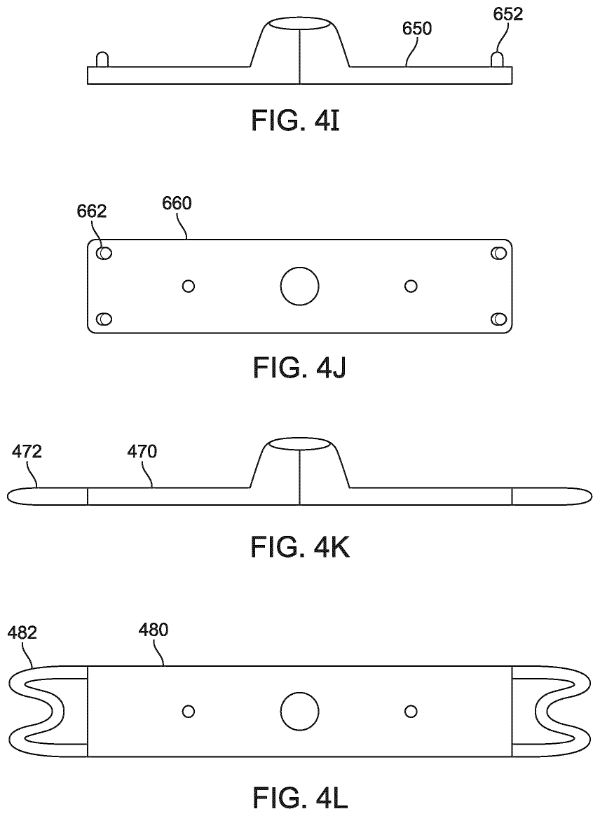

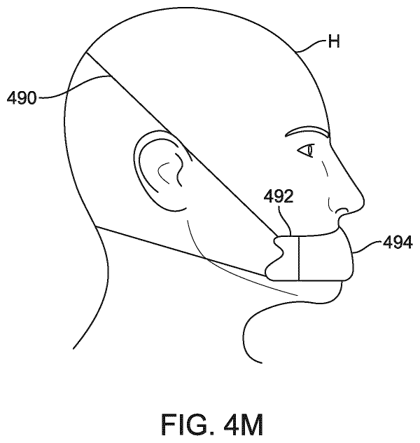

FIG. 4 shows a top view of the proximal end 202 of the endotracheal tube 200 in FIG. 2. A connector 216 is coupled to the proximal end of the elongate shaft 202 and allows the endotracheal tube 200 to be coupled to tubing attached to a ventilator or to any other device. Thus, air or other fluids are introduced into proximal aperture 214 and pass through a central lumen in the elongate shaft. The air then exits the distal aperture in the endotracheal tube thereby delivering the air to the patient's lungs. The mouth pad 210 is slidably disposed on the elongate shaft so that it may be moved into apposition with the mouth to form a seal, or moved away from the mouth to provide access to the oral cavity.

FIGS. 4A-4B show another example of a mouth pad 400 (also referred to as a mouth cover herein) that may be used with any of the examples of endotracheal tube disclosed herein. Mouth pad 400 is substantially the same as the mouth pad described in FIGS. 3-4 above with the major difference being that this mouth pad 400 includes one or more infusion ports 402, 404 that allow a fluid to be instilled or irrigated directly into the oral cavity (e.g. the mouth) thereby avoiding the need to move the mouth pad and deliver the fluid with a syringe or other irrigation device. Furthermore, the mouth pad 400 may remain in position apposed against the patient's mouth thereby further avoiding or eliminating the possibility of pathogens or aerosols escaping the mouth in addition to reducing exposure to bodily fluid splashed from the mouth. Another reason for including the port or ports 402, 404 is that this allows simultaneous introduction of the fluid into the mouth and removal of the fluid using one of the ports in the endotracheal tube (that will be discussed below) thereby reducing dwell time of the fluid in the mouth.

FIG. 4A shows a top view of the mouth pad 400 which is generally the same form as the mouth pad described in FIGS. 3-4. The mouth pad may include a semi-rigid region 406 to give support to the mouth pad and a resilient rim 408 that helps the mouth pad 400 conform to and appose the mouth and form a seal. The semi-rigid region 406 may be formed from any material such as a polymer like silicone or other materials known in the art. The resilient rim 408 also may be made from any material such as a polymer or a gel, or may be a non-latex material. In this example the mouth paid includes two irrigation ports 402, 404 but any number of ports may be used including 1, 2, 3, 4, 5, 6, 7, 8, 9, or more than ten ports may be used to introduce fluid into the mouth. As in FIGS. 3-4, mouth pad 400 also includes a slotted region 410 that allows any of the endotracheal tubes disclosed herein to be slipped into an aperture 412 in the mouth pad. Pulling or twisting opposite ends of the mouth pad will open the slotted region 410 providing access to the aperture 412. The endotracheal tube may then be slidably disposed through the aperture 412.

FIG. 4B shows a side view of the mouth pad 400 in FIG. 4A. The infusion ports, here two ports 402, 404 may be any shape or length but in this example one is cylindrical and the other is tapered. The ports may be rigid or resilient. Additionally, the ports may have any pattern of fenestrations 418 to achieve a desired flow pattern. For example, the fenestration pattern may be random or orderly. Flow may be constant along the length of the port or may vary as desired, or fluid velocity or pressure may be constant or varied along the port. Optionally, a connector such as a Luer connector 416 may be provided on the outer portion of the port to allow tubing, a syringe, or other device to be releasably coupled with the port so that the fluid may be introduced into the mouth. The fluid may be any of the fluids disclosed herein including water, saline, chorhexidine, or other therapeutic agents. A collar 420 may also be include on the outer surface of the mouth pad 400 to provide support and a strain relief for the endotracheal tube 414 once coupled with the mouth pad. The collar 420 may be formed from any material and may BE soft and resilient such as a polymer gel may provide.

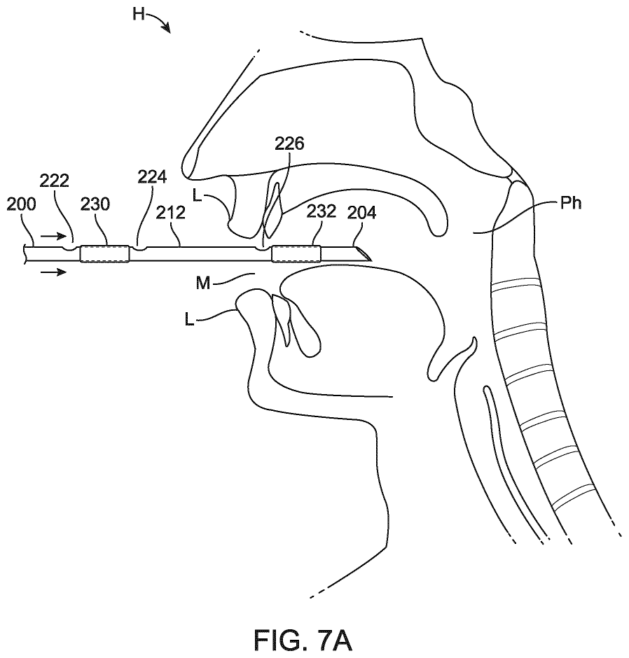

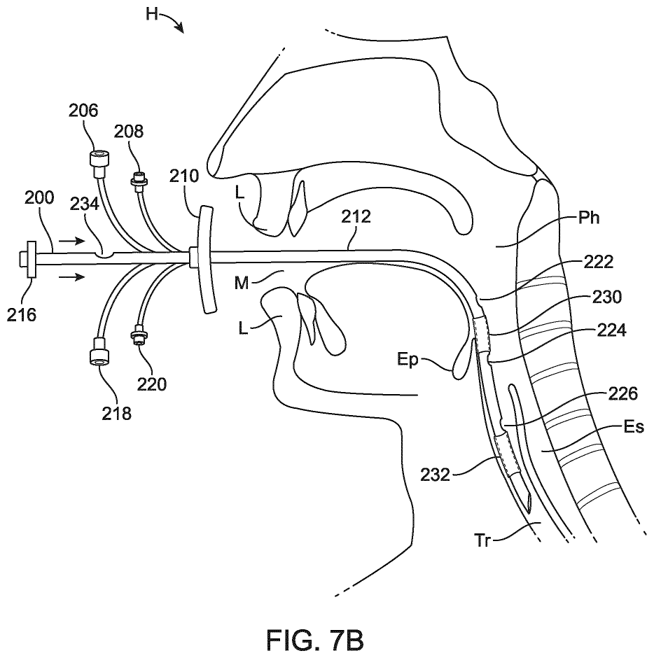

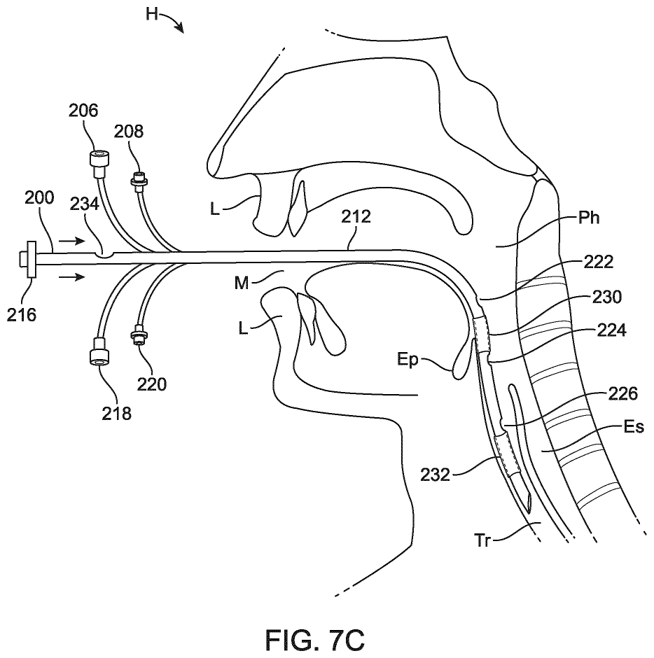

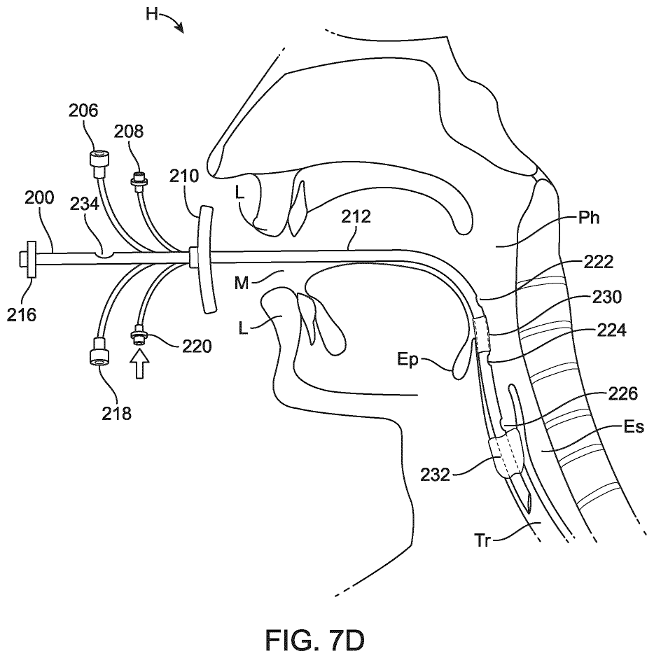

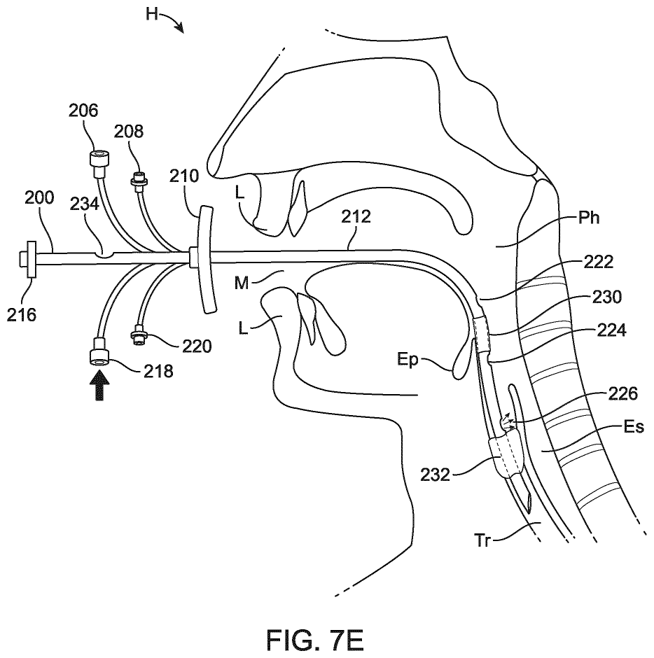

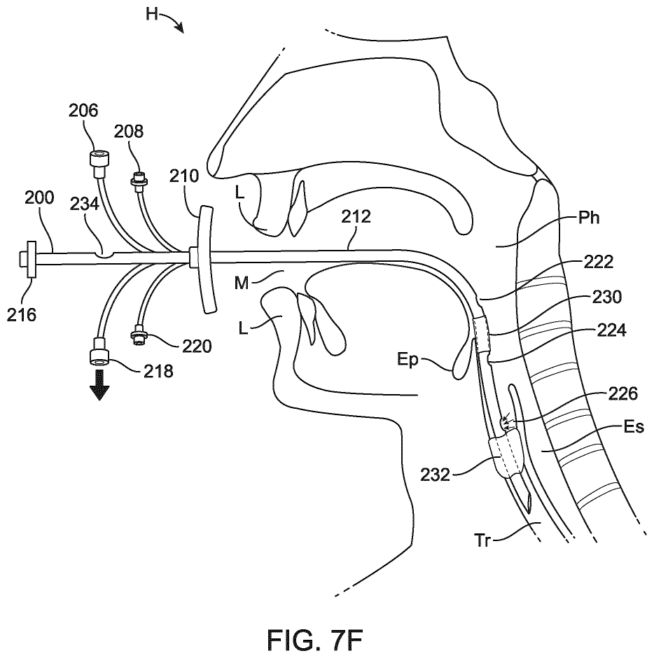

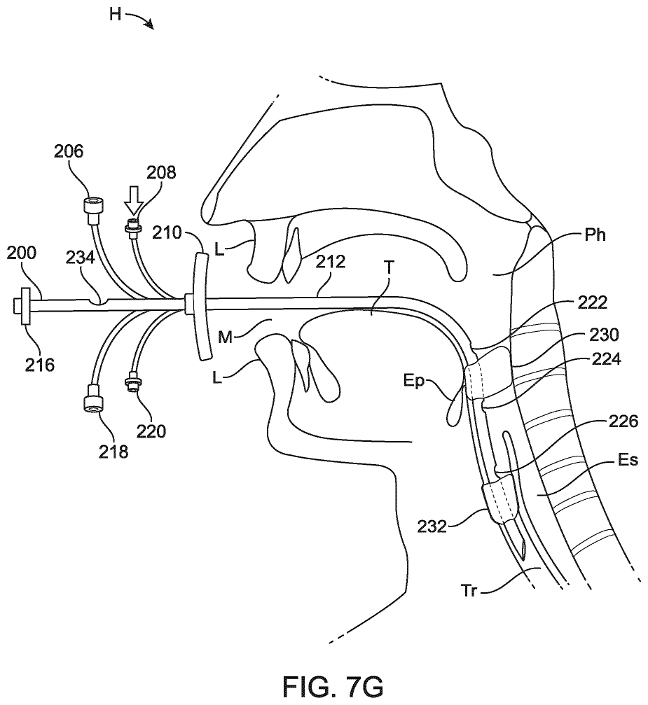

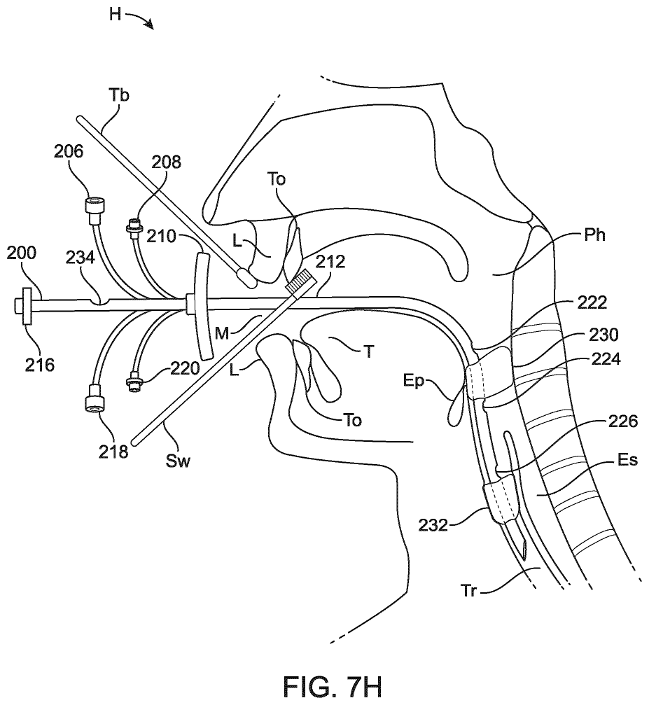

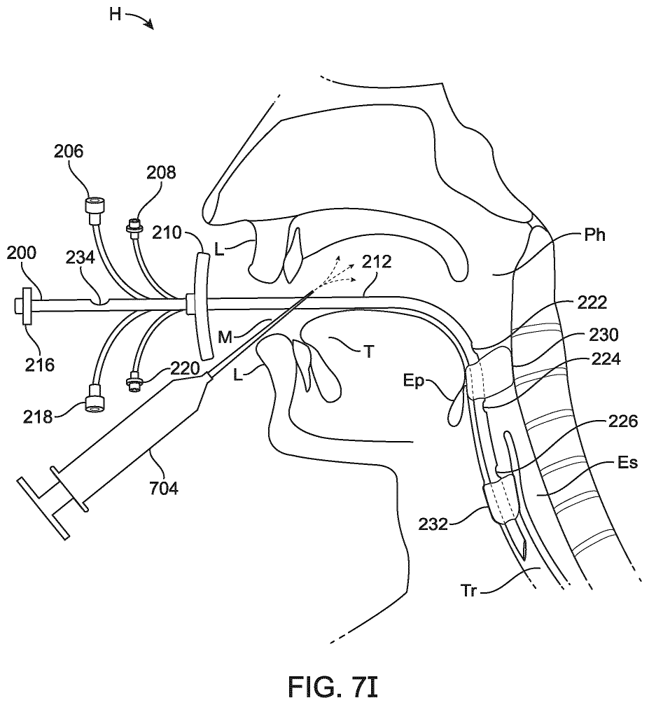

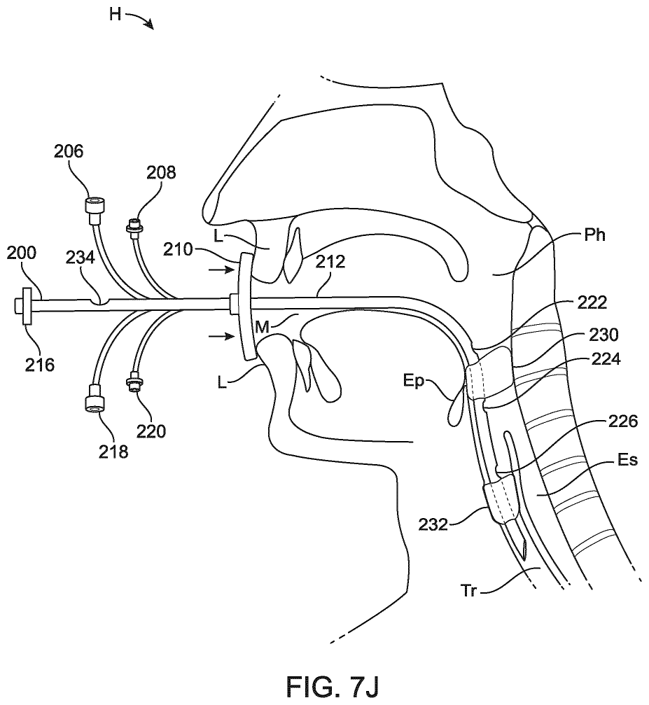

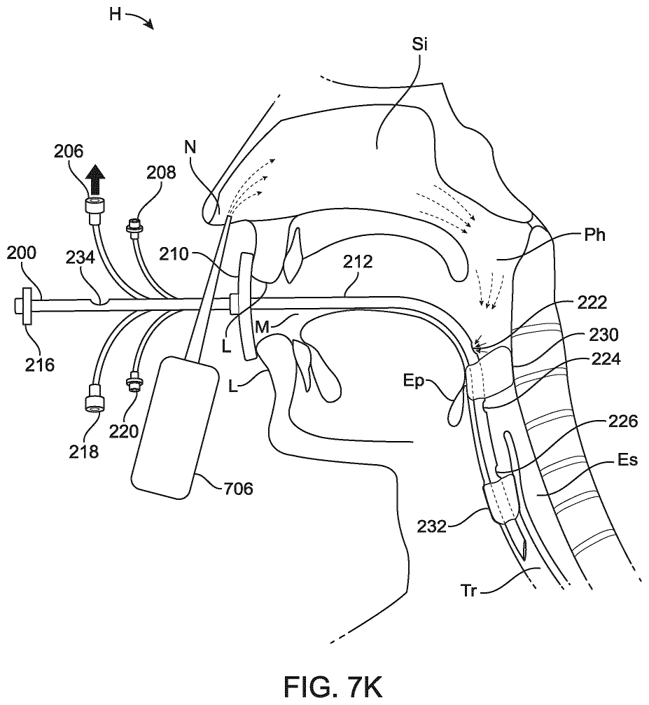

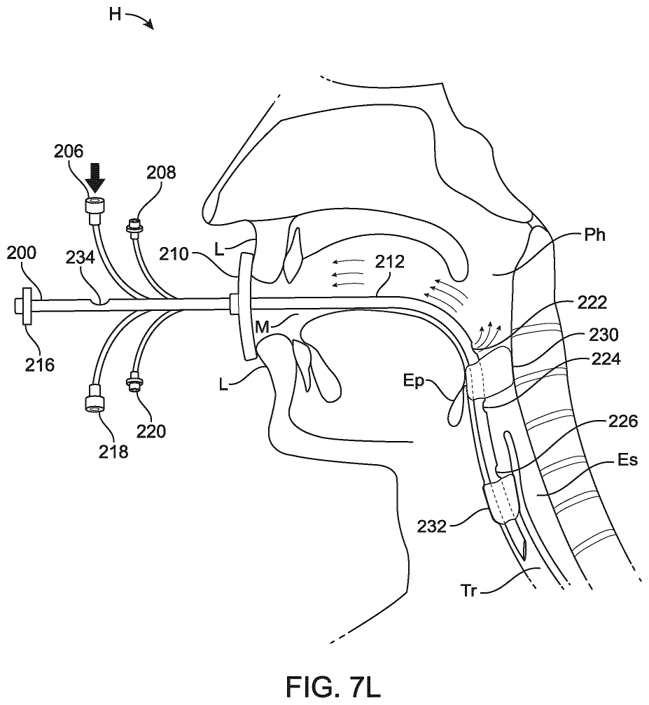

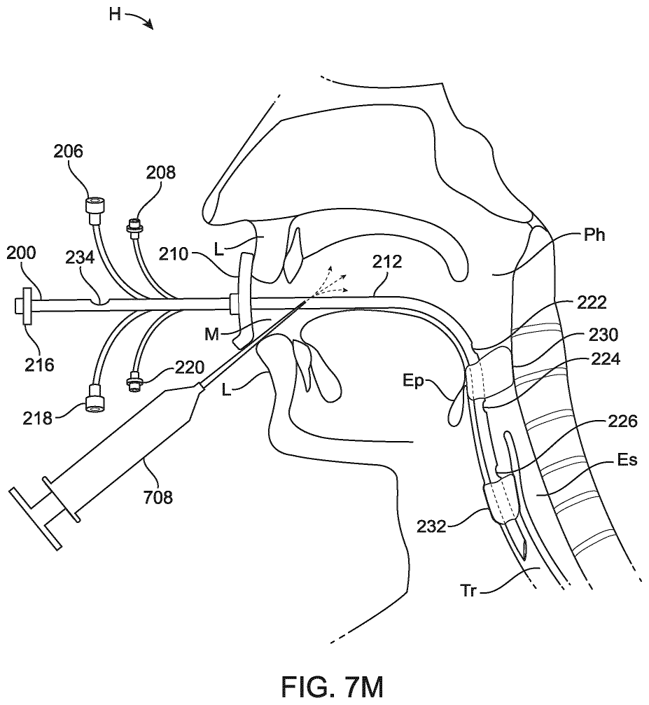

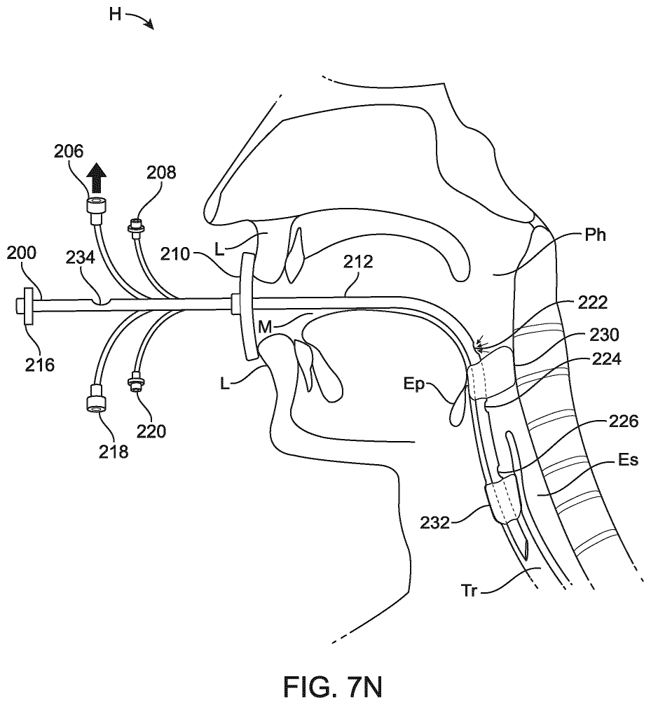

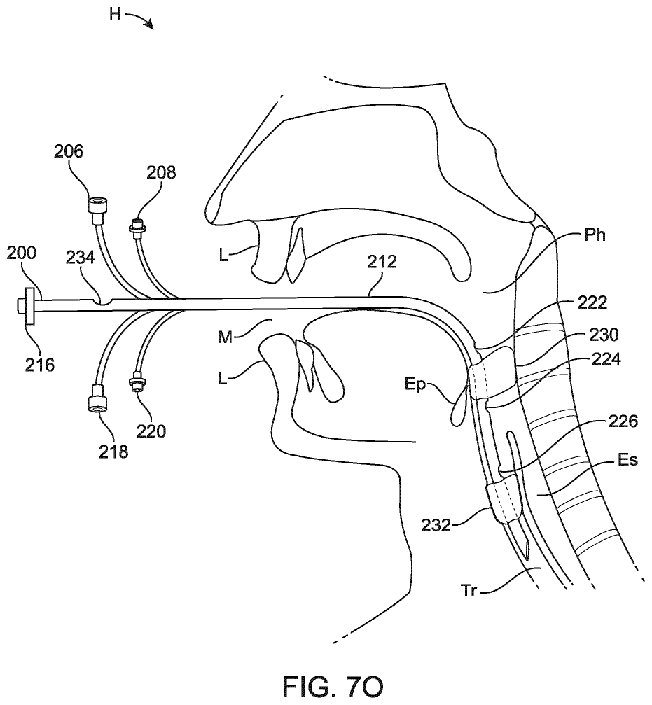

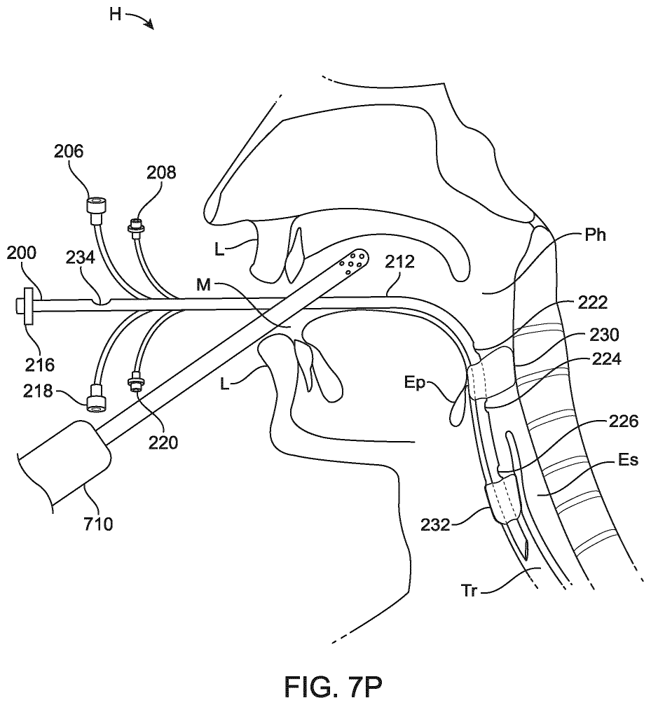

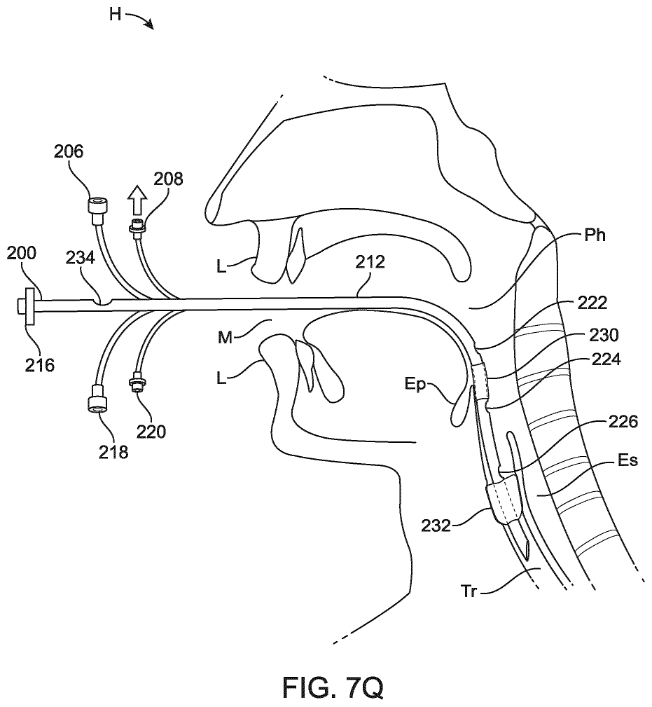

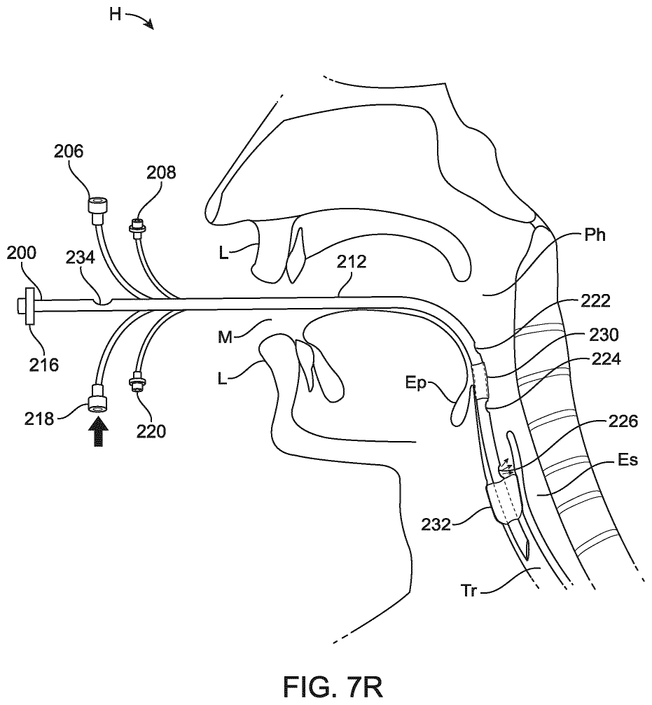

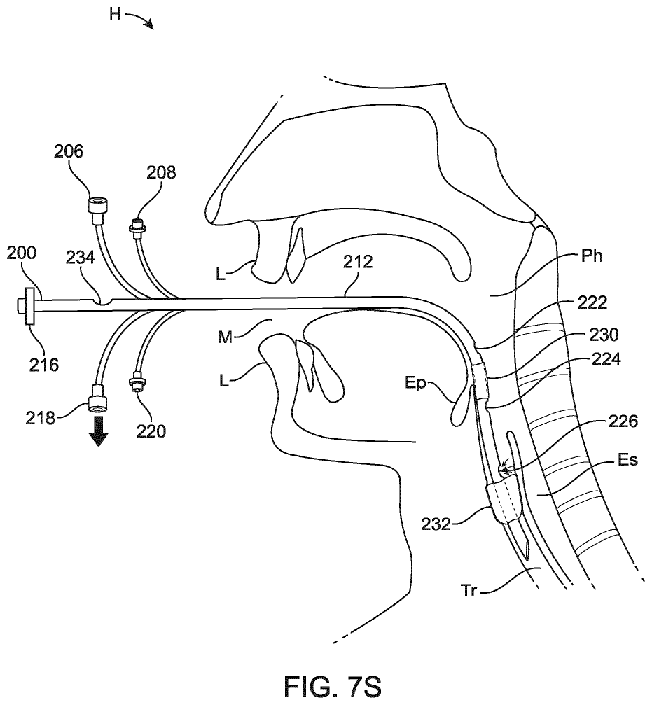

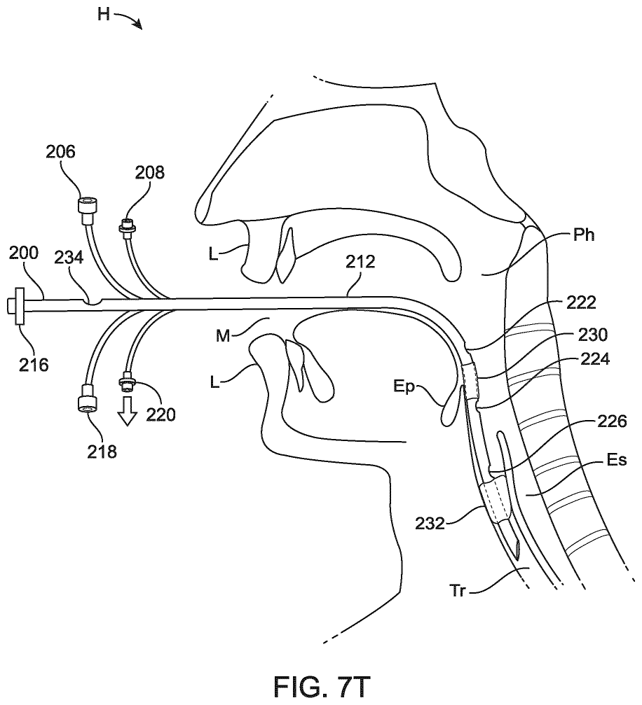

The mouth pad 400 of FIGS. 4A-4B may be used with any of the endotracheal tubes disclosed herein and in any of the methods described herein. Thus, whenever oral care is desired or needed, fluid may be irrigated or instilled into the oral cavity (e.g. the mouth) and the fluid may concurrently be removed via one of the ports on the endotracheal tube. For example, the port that is superior to the proximal-most expandable cuff may be used to remove the fluid and when the proximal-most balloon cuff is inflated this prevents the fluid from travelling further down the patient's throat. For example, the mouth pad 400 may be used to introduce fluid into the mouth in any of the oral care steps in FIGS. 7A-7U and removed via fenestration 222 (the fenestration proximal of expandable cuff 230) and collected with port 206. The nostrils may be pinched to ensure good suction from fenestration 222.

FIG. 4C shows that optionally, in any example of mouth pad, the ports 418 of FIGS. 4A-4B may not protrude from the mouth pad into the mouth and the connector such as Luer connector 416 may fluidly communicate via a through hole in the mouth pad directly into the patient's mouth. This provides a more streamlined profile for the mouth pad and may potentially reduce manufacturing costs for producing the mouth pad as and associated packaging which may otherwise need a recessed region to receive the protruding ports.

FIG. 4D shows an assembly view of an example of how a mouth pad 450 may be formed. The mouth pad 450 may include a mouth pad edge seal 452, a tube seal 456, a mouthpiece base 472 and a pad layer 470. The mouthpiece base 472 may be a rigid or semi-rigid flat and thin planar layer of material of any shape such as square, rectangular, oval, circular, or any other shape that conforms to a patient's mouth and adjacent areas. It optionally includes fluid infusion and suction ports 460 for the oral cavity and which may include a connector 460 such as a Luer connector on the anterior portion (e.g. facing away from the patient's mouth) to allow tubing, a syringe or another device to be coupled to the mouth piece, and flexible tubing 468 with a plurality of apertures to allow the fluid to irrigate the mouth or suction the fluid out of the oral cavity. The flexible tubing may be releasably coupled to the connectors 460 or they may be fixedly attached. Also, in any example of a mouth pad, the flexible tubing 468 may be replaced with the other examples of irrigation elements shown in FIGS. 4A-4C above. As previously descried above, the mouth pad 472 may include a seam 464 that can be separated when opposite ends of the mouth pad 472 are pulled away from on another to form a gap to allow the mouth pad to be slipped over any endotracheal tube being used on the patient including those disclosed herein as well as any commercially available device. Thus, the endotracheal tube will fit into the protruding portion 462 with cylindrically shaped channel. Optional loops 458 which may be wires, strings, filaments, or polymer tabs may be coupled to the anterior surface, posterior surface, or lateral surfaces of the mouth pad. These loops 458 allow a head band to be coupled to the mouthpiece to help ensure that the mouth pad remains secured to the patient, especially when the patient is pronated during ventilation. The loops may be any shape, here shown as sinusoidal, although other shapes such as arcs, circles, square, etc. may be used. A pad layer 470 is on the bottom of the assembly and therefore closest to the patient and engages the mouth. The pad is a soft and resilient layer such as a gel pad or a soft silicone material that conforms with the face to ensure a seal to prevent fluid or aerosols from escaping the mouth. The pad includes apertures 474, 476 to accommodate the irrigation/aspiration ports in the mouth pad in the layer above the pad, and similarly a split seam 478 allows the seam to be separated so the pad may be placed over the endotracheal tube. A tube seal 456 may be circumferentially wrapped around protrusion 462 and the endotracheal tube extending therefrom to help seal the two components together and prevent leakage of fluids or aerosols. Similarly, edge seal 452 may be placed around the perimeter 466 of the mouth pad 472 and cover portions of the face adjacent the perimeter of the mouth pad to further prevent leakage of fluids and aerosols. The edge seal 452 may be a band with a central aperture to allow the Luer connectors and endotracheal tube to pass through the central aperture and the edge seal may have the same geometry as the perimeter of the mouth pad. The edge seal and the tube seal may have an adhesive on one side to ensure that the seals stick to their adjacent substrate, and they may be soft and resilient to conform to the substrate.

FIG. 4E shows another example of a mouthpiece similar to the previous example in FIG. 4D with the major difference being that there are no sealing tapes. The mouthpiece 480 includes the mouth pad 482 which may be a flat planar and thin layer, and may be semi-rigid to provide support. The mouth pad 482 includes irrigation/aspiration ports 486 which may be any of the ports described in FIGS. 4-4D above for oral cavity cleaning, and a protruding portion 490 with a central cylindrically shaped channel for receiving an endotracheal tube. A slit 488 in the protrusion and pad allows the seam to be separated so that the endotracheal tube may be disposed in the cylindrically shaped channel. A resilient pad 484 is disposed under the mouth pad 482 and is soft and conforms to the patient's face and mouth in order to provide a seal and prevent leakage of fluids or aerosols from the patient. For example, a gel pad may be used for the resilient pad 484. Also, the gel pad may extend up and circumferentially around the cylindrically shaped channel in the protrusion 490 to help form a seal around the endotracheal tube, thereby also helping to prevent fluid or aerosol leakage.

FIG. 4F shows a side view of the example in FIG. 4E.

FIG. 4G shows another example of a mouthpiece 602 similar to the examples previously discussed but with the major difference being the inclusion of a flange that the patient may bite over to help anchor the mouthpiece and hold it in position. As before, the mouthpiece 602 includes a mouth pad 604 that may take any form or shape including a thin, flat rectangular pad that may be semi-rigid or resilient. An optional gel pad (not shown) may be disposed on the posterior portion (closest to the patient's mouth) to help seal the flat rectangular pad against the patient's face. Optional connectors 606 such as Luer connectors may be used to connect the mouth piece to tubing, syringes, or other fluid sources that can be used for oral hygiene and the fluid is delivered via ports 608 which may have any number of fenestrations so the fluid may be delivered and then suctioned out of the oral cavity. The ports may be any of those described herein. A protrusion 610 has a cylindrically shaped central channel 612 that receives the endotracheal tube (any of those disclosed or otherwise available) and a seam 614 allows the mouth pad to be separated so the endotracheal tube may be disposed in the central channel 612. A leg 616 on each side of the mouthpiece extends in the posterior direction into the patient's mouth and a flanged region 618 is disposed posterior to the teeth and helps secure the mouthpiece in the patient's mouth. Similarly, a flanged region 620 on a posterior portion of the protrusion 610 also fits behind the teeth and helps anchor the mouthpiece in the patient's mouth. The flanges act as anchoring elements. The patient's teeth fit in the gap between the posterior portion of the mouth pad 604 and the anterior portion of the flanges 618, 620.

FIG. 4H illustrates another example of an anchoring element that may be used to help hold a mouthpiece in the patient's mouth. The mouthpiece 626 includes a mouth pad 628 which may be the same as any of those previously described and may be a semi-rigid flat and planar element that fits against the patient's face with or without the optional gel pad. Connectors 632 such as Luer connectors may be coupled to the pad 628 and connected to tubing, a syringe, or another fluid source for oral cavity hygiene. Fluid introduced via connector 632 may be delivered into a port 640 with fenestrations such as any of those described herein to clean the patient's mouth and then suctioned out of the patient's mouth. A protrusion 636 from the pad 628 has a central channel 634 that holds the endotracheal tube. A seam 638 in the pad and protrusion may be split apart to allow the mouthpiece to be disposed over an endotracheal tube and placed into central channel 634. The mouthpiece also includes a soft resilient flange that extends from one side to the other side of the mouthpiece and this may be disposed behind the patient's lips and in front of the patient's teeth. The flange helps anchor the mouthpiece in the patient's mouth and also serves as a splash guard and helps minimize or prevent fluid or aerosol leakage out of the patient's mouth.

FIG. 4I shows an example of a mouthpiece which includes a fastener element that may be used to help secure the mouthpiece around the patient's head. The mouthpiece 650 may be any of those disclosed herein. A fastener element 651, here two, one on opposite sides of the mouthpiece may be used to help connect the mouthpiece to a headband. The fastener elements may be disposed on an anterior or posterior surface of the pad, and may be eyelets, rings, apertures, or any other fastener that may be coupled to a headband. The headband (not shown in this view) can then be wrapped around the patient's head to help secure the mouthpiece in the patient's mouth and against the patient's face to prevent fluid or aerosol leakage as well as preventing unwanted movement. The headband may be a strap, string, filament, or other strip of material. Other aspects of the mouthpiece may be the same as any example disclosed herein, including the protruding portion with central channel for holding an endotracheal tube and the seam that may be split to allow the endotracheal tube to be inserted into the central channel.

FIG. 4J shows still another example of a mouthpiece 660 with fasteners that can be used to help secure the mouthpiece to the patient. The mouthpiece 660 may be any of those disclosed herein. A fastener element 662, here four, one in each corner of the mouthpiece may be used to help connect the mouthpiece to a headband. The fastener elements may be disposed on an anterior or posterior surface of the pad and may be eyelets, rings, apertures, or any other fastener that may be coupled to a headband. The headband (not shown in this view) can then be wrapped around the patient's head to help secure the mouthpiece in the patient's mouth and against the patient's face to prevent fluid or aerosol leakage as well as preventing unwanted movement. The headband may be a strap, string, filament, or other strip of material. A single headband that has connection points on each side of the mouthpiece may be used, or two separate headbands may be coupled to the fasteners. Other aspects of the mouthpiece may be the same as any example disclosed herein, including the protruding portion with central channel for holding an endotracheal tube and the seam that may be split to allow the endotracheal tube to be inserted into the central channel.

FIG. 4K illustrates another example of a mouthpiece 470 which may be any of those described herein and includes a fastener for helping to secure the mouthpiece to the patient's head. Here, the fastener is a loop 472 on opposite lateral sides of the pad and this example includes two fasteners that can be coupled to a headband. The fastener elements may be eyelets, rings, apertures, or any other fastener that may be coupled to a headband. The headband (not shown in this view) can then be wrapped around the patient's head to help secure the mouthpiece in the patient's mouth and against the patient's face to prevent fluid or aerosol leakage as well as preventing unwanted movement. The headband may be a strap, string, filament, or other strip of material. A single headband that has connection points on each side of the mouthpiece may be used, or multiple separate headbands may be coupled to the fasteners. Other aspects of the mouthpiece may be the same as any exampled disclosed herein, including the protruding portion with central channel for holding an endotracheal tube and the seam that may be split to allow the endotracheal tube to be inserted into the central channel.

FIG. 4L shows another example of a mouthpiece 480 which may be any of those disclosed herein with a fastener 482 to help secure the mouthpiece to the patient. Here, the fastener is a loop with peaks and valleys and the fastener may be disposed on opposite lateral sides of the pad and this example includes two fasteners that can be coupled to a headband. The fastener elements may be eyelets, rings, apertures, or any other fastener that may be coupled to a headband. The headband (not shown in this view) can then be wrapped around the patient's head to help secure the mouthpiece in the patient's mouth and against the patient's face to prevent fluid or aerosol leakage as well as preventing unwanted movement. The headband may be a strap, string, filament, or other strip of material. A single headband that has connection points on each side of the mouthpiece may be used, or multiple separate headbands may be coupled to the fasteners. In this example, the fastener has two valley regions which may be used to ensure that a strap or other headband rests in the valley when in use and under tension after disposing the strap or headband around the patient's head. Thus, one or two or more straps or headbands may be used with the fastener. Other aspects of the mouthpiece may be the same as any exampled disclosed herein, including the protruding portion with central channel for holding an endotracheal tube and the seam that may be split to allow the endotracheal tube to be inserted into the central channel.

FIG. 4M shows a headband 490 which in this example includes two straps wrapped around the patient's head H. The free ends of the two straps are coupled to the fastener loop 492 that is included with the mouthpiece 494. The mouthpiece 494 may be any of the mouthpieces disclosed herein.

In addition to headbands, a band may be coupled to the fasteners on the mouthpiece and secured to the patient's neck, ears, head, or combinations thereof.

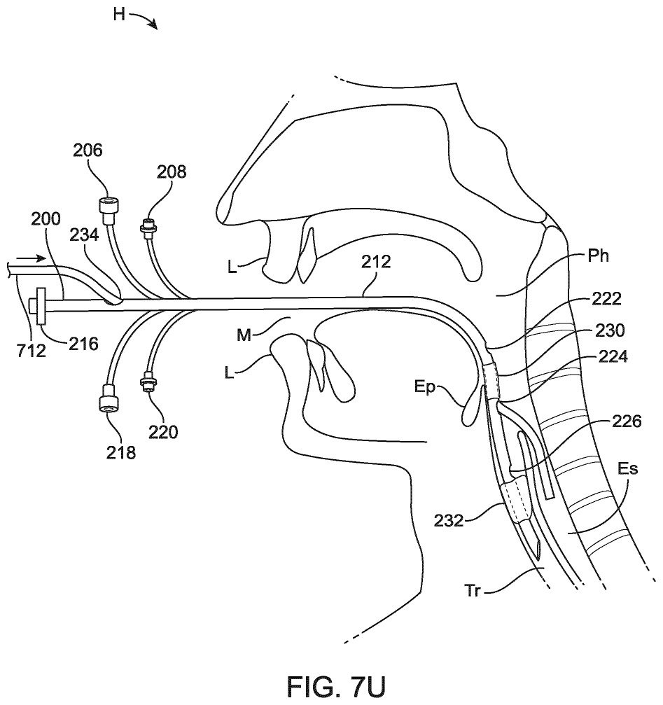

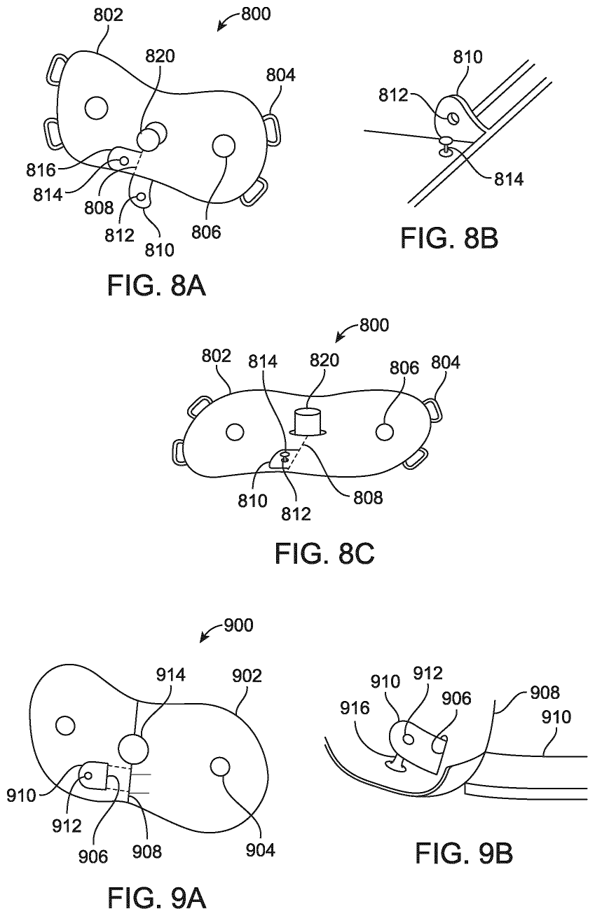

FIGS. 8A-8C illustrate another example of a mouth pad (also referred to as a mouth cover herein) that may be used with any example of endotracheal tube disclosed herein. The mouth pad 800 is similar to the mouth pads previously described above with the major difference being the optional fastener to help secure opposite ends or edges along a slit of the mouth pad together after the mouth pad has been inserted over an endotracheal tube. Here, the mouth pad 800 includes the substantially flat and planar pad 802 with fenestrations 806 for the irrigation/suction ports described above in FIGS. 4-4M above. In this example there are two fenestrations 806 for irrigation/suction ports as described previously, however, in some examples one fenestration may be for an irrigation/suction port, while the other fenestration may be used for an orogastric feeding tube to exit the mouth and mouth pad. Also, optionally the mouth pad may include any of the fasteners 804 described above in FIGS. 4-4M. Also as previously described above, a slit 808 in the mouth pad allows the mouth pad to be separated along the slit and then the endotracheal tube 820 is slid through the slit into the aperture in the mouth pad. In some situations, it may be advantageous to provide a fastener that can help secure opposite ends along the slit 808 together so the slit does not reopen and allow the endotracheal tube 820 to separate from the mouth pad. Here, the fastener may include a tab 810 with an aperture 812 through the tab on one side of the slit. The other side of the slit may include a protuberance 814 such as a pin with a narrow elongate pin and an enlarged head. A recessed region 816 may also be disposed in the mouth pad to receive the tab. After the mouth pad has been disposed over the endotracheal tube and the slit closes, the tab 810 may be engaged with the protuberance so that the two halves of the mouth pad remain coupled together. In some examples the pin and aperture fastener may be replaced with a hook and loop fastener like VELCRO with the hooks on either the tab or the mouth pad, and the loops on the other of the tab or mouth pad. Similarly, in any example of a fastener disclosed herein, the fastener described may be replaced with a hook and loop fastener.

FIG. 8B shows a close-up of the tab and protuberance that form the fastener in mouth pad 800 of FIG. 8A. Here, the tab 810 includes aperture 812 that can be disposed over protuberance 814 which includes an elongate pin and an enlarged head that can fit through aperture 810 but does not easily escape from aperture 810 (or a hook and loop fastener may be used). The protuberance and the tab are on opposite sides of the slit in the mouth pad.

FIG. 8C shows mouth pad 800 in FIG. 8A with the fastener closed. Here, tab 810 has been disposed across slit 808 and the aperture 812 in tab 810 has been disposed over protuberance 814 thereby securing both halves of the mouth pad separated by the slit together. As previously mentioned, the fastener may be a hook and loop fastener instead of the pin and aperture. Other aspects of the mouth pad 800 may generally take the form of any of the examples in FIGS. 4-4M above. Any of the examples of the mouth pad disclosed herein may include the fastener described in FIGS. 8A-8C.

FIGS. 9A-9B show another example of a fastener that may be used in any of the examples of mouth pads disclosed herein. Mouth pad 900 in FIG. 9A is generally the same as the mouth pad in FIGS. 8A-8C above with the major difference that the mouth pad includes another slit for the fastener tab to pass through to further secure the opposite ends of the mouth pad across the slit together. This is similar to a belt buckle concept.

In FIG. 9A the mouth pad 900 includes a flat planar pad 902 similar to the mouth pads previously describe above. An aperture 914 is sized and shaped to receive any of the endotracheal tubes described herein. Slit 908 allows the mouth pad to be opened up so that it can be slid over the endotracheal tube so that the endotracheal tube fits in aperture 914. Irrigation and suction fenestrations 904 are generally the same as those previously described above. Therefore, both fenestrations 904 may be for suction/irrigation, or one may be for suction/irrigation and the other for an orogastric feeding tube The fastener is generally the same as the fastener previously described above in FIGS. 8A-8C with a tab 910 having an aperture 912 that can fit over a pin 916 (best seen in FIG. 9B) with an enlarged head. In this example, the mouth pad optionally incudes an additional slit 906 in the mouth pad so that the tab can fit through the slit as it crosses the slit region 908 to be coupled to the pin 916. The additional slit 906 provides additional stability to the tab to prevent it from unbuckling from the pin, thereby providing a secure fastener to keep both ends of the mouth pad coupled together and preventing the endotracheal tube from slipping out of aperture 914.

FIG. 9B shows the fastener from mouth pad 900 in FIG. 9A more clearly. Here, tab 910 passes through slit 906 which is disposed in the mouth pad on the opposite side of the slit 908. The aperture 912 in tab 910 can then be disposed over a protuberance 916 which includes an elongate pin and an enlarged head region that prevents the pin from slipping out of aperture 912.

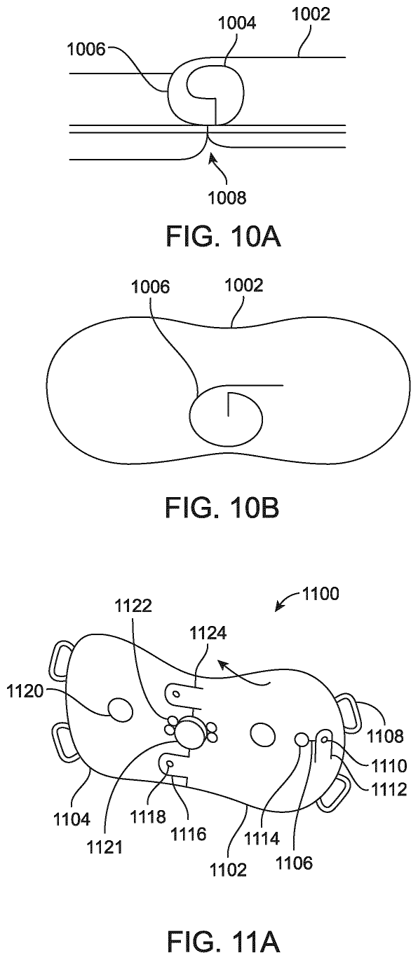

FIG. 10A shows another example of a fastener that may be used in any of the examples of mouth pads to help secure the ends of the mouth pad together that are separated by the slit. Here, mouth pad 1002 includes two halves that are separated by slit 1008. When the two halves are pulled away from one other the slit opens up and allows the slit to be passed over an endotracheal tube including any of those disclosed herein, so that the endotracheal tube can be disposed in the center aperture of the mouth pad (not shown). Here, opposite sides of the mouth pad may include tabs that lock with one another. Here tab 1006 on one side of the mouth pad and tab 1004 on the other side of the mouth pad, separated by the slit 1008, can be interlocked with one another to prevent the slit from separating and enlarging thereby preventing the endotracheal tube from being released from the mouth pad. Here, the tabs include arcuate head regions that interdigitate with one another to coupled them together.

FIG. 10B shows another view of the locking tabs in FIG. 10A. Other aspects of FIG. 10B are generally the same as any of the other examples of mouth pads disclosed herein.

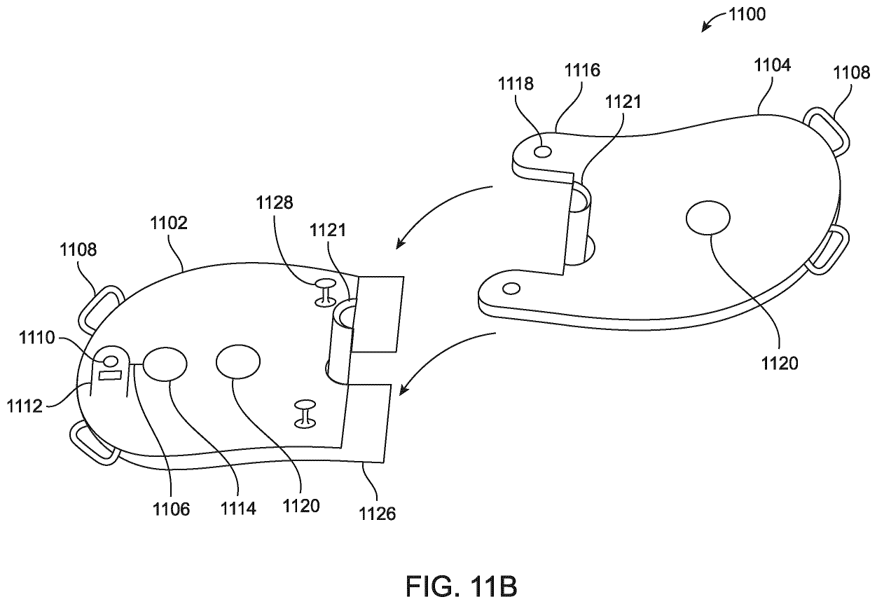

FIGS. 11A-11B show an example of a two-piece mouth pad that may be used with any of the endotracheal tubes disclosed herein. Additionally, any of the features from any of the mouth pads disclosed herein may be used in combination with the features disclosed in this example or substituted with any of the features disclosed in this example.

In FIG. 11A, the mouth pad 1100 includes two discrete portions 1102, 1104 that are coupled together with one or more fasteners. This allows the halves to be easily disposed around the endotracheal tube (not shown in this view) which may be any of the endotracheal tubes disclosed herein. The two halves are then fastened together using a fastener, here two fasteners that include a tab 1116 with an aperture 1118 that extends across the seam 1124 and is disposed over a pin with an enlarged head on the other half of the mouth pad to hold the tab on the pin, similar to the fastener previously described above. The fastener may also be a hook and loop fastener. Thus, the two fasteners keep the two halves coupled together and form a seal that either minimizes or prevents aerosols or other droplets from escaping the patient's mouth. Other features of the mouth pad are similar to those previously described, such as a fastener 1108 for helping secure the mouth pad to the patient's head or neck, here there are four fasteners to which a strap may be attached. Also, there is an aperture 1121 for the endotracheal tube which may be any of those disclosed herein. Fenestrations 1120, here two, allow the irrigation and suction ports to be coupled to the mouth pad as previously described. This example also includes an aperture 1114 for an orogastric tube (not shown in this view) and a slit 1106 extends from the aperture 1114 to the edge of the mouth pad. The opposite edges of the mouth pad along slit 1106 may be separated from one another to allow the orogastric tube to be passed through the slit and into aperture 1114. A fastener may then be used to couple the edges of the slit together, such as with a tab 1112 that passes over the slit 1106 so that an aperture 1110 in the tab 1112 can be disposed over a pin with an enlarged head on the other edge of the mouth pad to hold the two edges together and prevent the orogastric tube from slipping out of aperture 1114. The fastener for the orogastric tube may also be a hook and loop fastener. In another example, instead of two fenestrations 1120 for suction/irrigation, the mouth pad may only have one fenestration 1120 and one aperture 1114 for the orogastric tube if the mouth pad is small and surface area is limited.

Additionally, in previous examples, the irrigation (also referred to herein as instillation), suction, and cuff inflation lines were illustrated as being coupled to the endotracheal tube at a position outside the oral cavity and adjacent a proximal end of the endotracheal tube. However, in this example the irrigation, suction and cuff inflation lines may be coupled to the endotracheal tube along a portion of the endotracheal tube that is disposed in the oral cavity, behind or distal of the mouth pad and proximal of the upper inflation cuff. This allows manipulation of the lines with ease and reduces the risk of pulling on the lines that could move the endotracheal tube which could dislocate the tube and compromise the patient's airway.

Therefore, in this example, the mouth pad may include one or more fenestrations 1122, here four, that allow the four irrigation, suction, and cuff inflation lines to be passed through the apertures 1122 to exit the oral cavity. Therefore, instead of the lines exiting the mouth and being sandwiched between the mouth pad and the patient's lips which may not provide an adequate seal against droplets and aerosol. However, this is not intended to be limiting and any of the lines may still exit the patient's mouth and be disposed between the mouth pad and the patient's lips without passing through an aperture in the mouth pad.

FIG. 11B more clearly illustrates the two-piece mouth pad 1100 example of FIG. 11A. The mouth pad portions which may be halves may each include a thin section of pad 1126 that extend under or over the adjacent edge of the other half of adjacent mouth pad half. Thus, when coupled together, this helps seal the seam to prevent aerosols or droplets from passing through the seam. The elongate pin 1128 with enlarged head is also more clearly shown in this figure, and this is inserted into the aperture 1118 in the tab 1116 to secure the two halves of the mouth pad together. The fastener may also be a hook and loop fastener. The aperture 1121 for the endotracheal tube may optionally include a raised collar with half the collar on one part of the mouth pad, and the other half of the raised collar on the other part of the mouth pad that helps support and provide a strain relief for the endotracheal tube when disposed in the aperture 1121, as well as sealing around the endotracheal tube to prevent droplets or aerosols from exiting the patient's mouth. The apertures 1122 for the irrigation, suction and inflation lines may be included in the mouth pad, or they may be omitted. FIG. 11B does not show these apertures for convenience. Other aspects of FIG. 11B are the generally the same as in FIG. 11A and are not repeated for the sake of brevity.

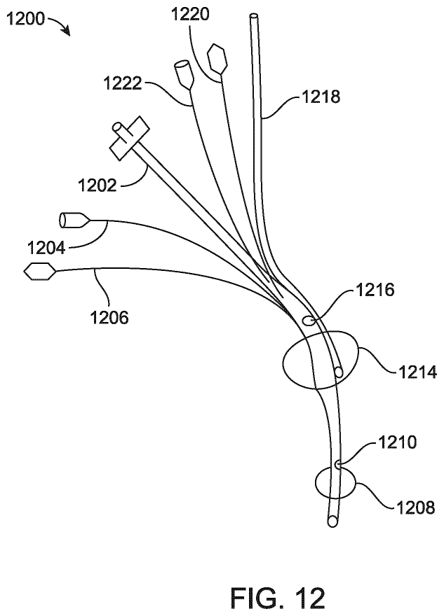

FIG. 12 illustrates another example of an endotracheal tube without the mouth pad attached. The mouth pad may be any of the example of mouth pad disclosed herein. As disclosed above in other examples of endotracheal tubes, the irrigation, suction, or inflation lines are shown coupled to the endotracheal tube adjacent the proximal end of the endotracheal tube. Therefore, the lines are all coupled to a position on the endotracheal tube that is outside the patient's oral cavity and proximal of the mouth pad. However, in some situations is may be advantageous to have some or all the lines coupled to the endotracheal tube at a point along the endotracheal tube that is in the oral cavity and therefore distal of the mouth pad. As previously mentioned, this facilitates manipulation of the lines with ease and also reduces the risk of pulling on one or more of the lines in a direction that could dislocate the endotracheal tube and comprise the patient's airway.

FIG. 12 shows an example where all the lines are coupled to the endotracheal tube distal of the proximal end of the endotracheal tube and proximal of the proximal-most inflatable cuff. Thus, the coupling position is along a portion of the endotracheal tube that is disposed in the patient's oral cavity and behind the mouth pad. For example, endotracheal tube 1200 includes the subglottic suction line 1204, subglottic inflation line 1206, proximal pharyngeal suction line 1222 and proximal inflation line 1220 which generally take the same form as previously described in this specification. However, they connect with the endotracheal tube at a position along the endotracheal tube that is distal of the mouth pad and proximal of the proximal cuff and therefore this position is along a portion of the endotracheal tube that is disposed in the patient's oral cavity. This helps with line management and also helps minimize the possibility that the lines are disposed distally of the glottis which is undesirable and minimizes the possibility of pulling on one of the lines and dislodging the endotracheal tube and compromising the airway. Because the lines are coupled to the endotracheal tube behind or distal of the mouth pad, apertures or fenestrations in the mouth pad may be used to ensure that the lines can easily pass through the mouth pad through the apertures for access by a healthcare provider, such as in the examples of FIG. 11A. The apertures may provide a relatively tight seal against the lines to prevent droplets or aerosols from escaping the patient's mouth, which may be more desirable than trying to seal the mouth pad against the patient's mouth with lines running therebetween which can leave gaps for aerosols or droplets to escape. In other examples, one or more of the lines may exit the patient's mouth and pass under the mouth pad, between the mouth pad and patient's lips instead of passing through the apertures, if desired.

Additionally, in order to facilitate insertion of an optional orogastric feeding tube into the endotracheal tube, a separate tube or line 1218 may be coupled to the aperture on the endotracheal tube which provides access to the orogastric lumen in the endotracheal tube. The separate orogastric line may enter the aperture on the endotracheal tube and extend through the orogastric lumen and terminate at or exit the orogastric lumen at the exit aperture on the endotracheal tube which is slightly distal of the upper cuff 1214. Thus, an orogastric feeding tube may be easily inserted into the line 1218 and advanced distally along the line through the endotracheal tube and out the endotracheal tube into a desired position distal of the proximal cuff. The additional line for the orogastric tube may have a rigid distal portion adjacent the proximal cuff 1214 to ensure that the orogastric tube may be properly positioned and to prevent line kinking, and the other portions of the additional line may be resilient as desired to provide flexibility. Thus, the line 1218 may have different regions of flexibility or resilience. Other aspects such as the subglottic cuff 1208, subglottic suction fenestration 1210, proximal cuff 1214, proximal suction fenestration 1216 generally take the same form as described in this specification.

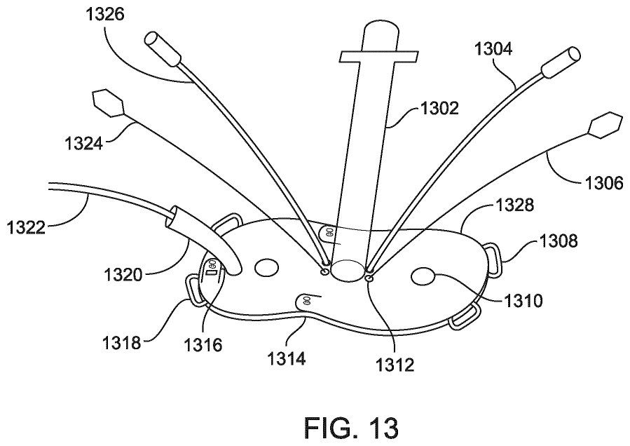

FIG. 13 shows the endotracheal tube of FIG. 12 combined with the mouth pad of FIG. 11A. The endotracheal tube 1302 includes a proximal pharyngeal suction line 1304, a proximal cuff inflation line 1306, a subglottic suction line 1326 and a subglottic inflation line 1324. These four lines are all coupled to the endotracheal tube behind or distal of the mouth pad 1328 and proximal of the subglottic inflation cuff (not shown), therefore they are coupled to the endotracheal tube at a position that lies in the patient's oral cavity. The lines exit the patient's mouth and pass through the apertures in mouth pad. However, as previously disclosed, some or all of the lines do not have to pass through the mouth pad apertures and they may still exit the patient's mouth and pass between the mouth pad and the patient's lips if desired. The mouth pad may be resilient enough to still seal around the lines and prevent droplets or aerosols from escaping the patient's mouth.

An orogastric line 1320 is also coupled to the endotracheal tube 1302 that allows an orogastric feeding tube 1322 to be easily inserted into the line 1320 and through the endotracheal tube and out the endotracheal tube into a desired position in the patient. Again, the orogastric line 1320 may have a rigid section adjacent the proximal inflation cuff and the rest of the line may be more resilient. Other aspects of the endotracheal tube generally take the same form as disclosed herein.

A mouth pad 1328 is also coupled to the endotracheal tube 1302. The mouth pad includes fenestrations 1312 that allow the four lines to pass through the mouth pad. Apertures 1310 allow coupling to suction and irrigation lines for the oral cavity as described herein. A slit (not shown) allows the mouth pad to be loaded over the endotracheal tube and then a fastener such as a tab 1314 that locks onto a post as previously discussed may be used to secure opposite edges along the seam, or a hook and loop fastener may be used. A second slit region 1316 similarly allows the mouth pad to be loaded onto the orogastric line 1320 and another fastener such as a tab 1318 that locks onto a pin helps keep that seam closed, or a hook and loop fastener. The orogastric feeding tube 1322 may be advanced through line 1320. Fasteners 1308, here four, allow straps or other filaments to be coupled to the mouth pad to help secure the mouth pad to the patient's head or neck. Other aspects of the mouth pad generally take the same form as disclosed herein. Again, the example in FIG. 13 shows two apertures 1310 for suction/irrigation and one aperture that accommodates the orogastric feeding tube. In some examples, there may only be one aperture 1310 for suction/irrigation and one aperture for the orogastric feeding tube.

In the examples described herein, many of the mouth pads have a long axis and a short axis and the slit is generally along the short axis of the mouth pad. However, one of skill in the art will appreciate that this is not intended to be limiting and the slit may be rotated approximately 90 degrees so that the slit runs along the long axis of the mouth pad if desired. The slit may also have any orientation relative to the long or short axis of the mouth pad, as needed.

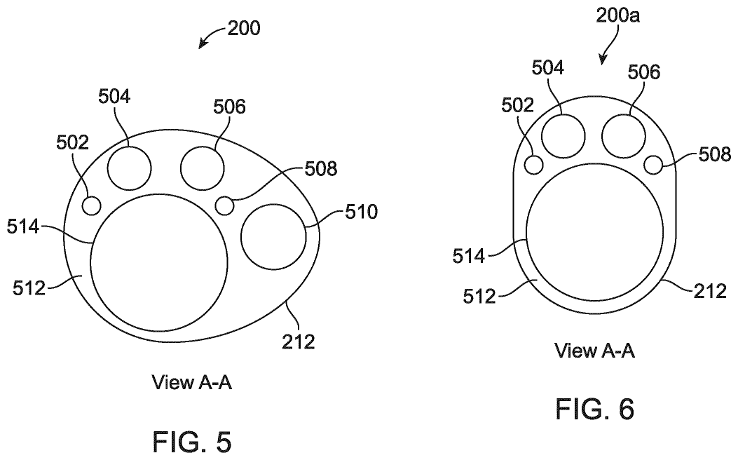

FIG. 5 is a cross-section of the endotracheal tube 200 taken along the line A-A in FIG. 2. In this example, the endotracheal tube includes six lumens. Two inflation/deflation lumens for the expandable cuffs, a central lumen for the air delivered to the lungs, and three lumens for the fenestrated regions in the elongate shaft. Each lumen is separate and discrete from the other lumens and provides an independent path. The endotracheal tube 200 includes elongate shaft 212 with lumens extending along the longitudinal axis of the elongate shaft 212. The central lumen 514 for air provided by a ventilator and delivered to and from the lungs may extend the entire length of the elongate shaft 212. An inflation/deflation lumen 502 may extend from proximal port 220 to the lower expandable cuff 232 best illustrated in FIG. 2. Fluid such as air or saline may be delivered along lumen 502 to inflate or deflate the lower cuff 232 to form a seal in the trachea. Similarly, a second inflation/deflation lumen 508 may extend from proximal port 208 to the upper expandable cuff 230 seen in FIG. 2. Fluid such as air or saline may be delivered along lumen 508 to inflate or deflate upper cuff 230 to form a seal in the pharynx or hypopharynx or region above the vocal chords in the larynx. Both lumens 502 and 508 are disposed in a wall 512 of elongate shaft 212.

Lumen 504 also extends along the longitudinal axis of the elongate shaft 212 and is fluidly coupled with port 218 and fenestration 226. Thus, fluid such as saline or an antiseptic or other therapeutic agents may be introduced into port 218 and delivered via fenestration 226 to the subglottic region superior to the lower inflation cuff 232. After the fluid is delivered, it may be removed by aspirating it out of fenestration 226 and out of port 218 into a syringe or other collection device. When the lower expandable cuff is inflated, this prevents the fluid from passing distal of the cuff into the lungs. Lumen 504 may be any size or shape depending on the desired flow requirements and is disposed in the sidewall 512 of elongate shaft 212, however in this example the lumen has a circular cross-section.

Lumen 506 extends along the longitudinal axis of the elongate shaft 212 and is fluidly coupled with port 206 and fenestration 222. Thus, fluid such as saline or an antiseptic or other therapeutic agent may be introduced into port 206 and delivered via fenestration 222 to the pharyngeal or hypopharyngeal region or region above the vocal chords, superior to the upper expandable cuff 230. After the fluid is delivered, it may be removed by suctioning it out of fenestration 222 and out of port 206 into a syringe or other collection device. When the upper expandable cuff is inflated, this prevents the fluid from passing distal of the cuff further into the trachea or esophagus. Lumen 506 may be any size depending on the desired flow requirements and is disposed in the sidewall 512 of elongate shaft 212, however in this example has a circular cross-section.

The inflation/deflation lumens 502, 508 may be smaller than the instillation/suction lumens 504, 506 since lower flow rates are required for cuff inflation and if a gas such as air is used for inflation it has a lower viscosity than most liquids thereby permitting the smaller lumen size.

A lumen 510 that may be larger than the lumens 504, 506 is also disposed in the sidewall 512 of elongate shaft 212. This lumen is optional and is coupled with proximal fenestration 234 and distal fenestration 224. This lumen is optionally used to allow an orogastric feeding tube to be inserted outside the patient's mouth via fenestration 234, inserted through the lumen 510 and exit fenestration 224 where the feeding tube can then be guided into the esophagus and into the stomach.

Lumen 510 may be any size or shape but in this example has a circular cross-section that is sized to slidably receive an orogastric feeding tube.

The cross-section of elongate shaft 212 may also be any size or shape to accommodate all the lumens but should be small enough to be disposed through the patient's mouth down the throat into the trachea. And in this example, the cross-section is an oblong oval with asymmetrical shape.

FIG. 6 shows the same cross-section as FIG. 5 but with the optional orogastric lumen removed from the endotracheal tube 200a. Other features are generally the same, and thus are labelled with the same reference numbers as FIG. 5. In addition to removal of the orogastric lumen, the corresponding upper and lower fenestrations 224, 234 would also be removed from the elongate shaft that is shown in FIG. 2. Any cross-section may be used but because one less lumen is used in this example, the cross-sectional profile is reduced and, in this example, an oval cross-section is illustrated.

In either example shown in FIG. 5 or FIG. 6 the elongate shaft may be formed from materials that are rigid enough to be inserted into the patient yet resilient and malleable enough to conform with the curves and bends of the native anatomy. For example, polyvinyl chloride (PVC), polyurethane, silicone, etc. may be used.

FIGS. 7A-7U illustrate an example of a method of intubating a patient that may reduce the incidence of VAP.

Prior to inserting the endotracheal tube in the patient, the patient should be correctly positioned such as sitting partially upright or semi-recumbent, or in a supine position. Correct position will depend on the individual patient and the operator performing the endotracheal insertion procedure. Additionally, appropriate sedatives are given to the patient. Oral and nasal irrigation may occur once the patient has been stabilized.

FIG. 7A shows the endotracheal tube 200 of FIG. 2 being inserted past the patient's lips L into the mouth M and advanced distally toward the pharynx Ph. Other aspects of endotracheal tube 200 including the elongate shaft 212, cuffs 230, 232, fenestrations 222, 224, 226 and distal end 204 are generally the same as previously described above in FIG. 2. The anatomy of the head H is the same as previously described in FIG. 1 and several reference numbers have been omitted for reader convenience. Both cuffs 230, 232 are in the deflated or collapsed configuration. Of course, one of skill in the art will appreciate that if the endotracheal tube has already been placed then the intubation steps may be omitted, and the operator may proceed directly to the oral and nasal cleaning steps described below.

FIG. 7B shows further distal advancement of the endotracheal tube 200 as indicated by the arrows. Here the endotracheal tube 200 has been advanced so that the distal end is disposed in the trachea Tr. The lower expandable cuff 232 is also disposed in the trachea Tr, and fenestration 226 is disposed adjacent the lower expandable cuff 232 and superior to the lower cuff, but still in the trachea Tr, also referred to as the subglottic region. The endotracheal tube does not extend into the esophagus Es. The upper expandable cuff 230 may be disposed in the pharynx Ph or just below the pharynx in the hypopharyngeal region or in the region above the vocal chords in the larynx. In this example the upper cuff 230 is disposed in the hypopharyngeal region and adjacent with the epiglottis Ep. Fenestration 224 is disposed in the hypopharyngeal region and inferior to the upper cuff 230, although it may also be disposed in the pharynx or the region above the vocal chords in the larynx. Fenestration 222 is also disposed in the hypopharyngeal region although it may be in the pharynx or in the region above the vocal chords in the larynx and is superior to the upper cuff 230. Both upper and lower cuffs are deflated during the insertion procedure.

Ports 206, 208, 218, 220 are disposed adjacent the lips L, but outside the mouth M. Fenestration 234 is also outside the mouth M along with connector 216. The optional mouth pad 210 is coupled to the elongate shaft 212 although it may be coupled later in the procedure as will be discussed further. Any of the mouth pads disclosed herein may be substituted for mouth pad 210. For example, mouth pad 400 may be used. For convenience, the rest of FIGS. 7D-7N are illustrated with the mouth pad coupled to the elongate shaft, but one of skill in the art will appreciate that the mouth pad is optional and may not need to be coupled to the elongate shaft. The endotracheal tube 200 is now correctly positioned in the patient. All other aspects of the endotracheal tube are generally the same as in FIG. 2, and all other aspects of the anatomy are generally the same as FIG. 1.

FIG. 7C shows the same step in the procedure and same view as FIG. 7B but with the optional mouth pad 210 removed or other mouth pad such as mouth pad 400 removed. All other aspects of FIG. 7C are the same as FIG. 7B.

FIG. 7D shows expansion of the lower cuff 232 on the endotracheal tube 200. Here, fluid such as a gas like air, or a fluid like water or saline is injected into port 220 as indicated by the arrow where it flows through its own discrete lumen to inflate the lower expandable cuff 232 into apposition with wall of the trachea Tr. Here, a tight seal between elongate shaft 212 and the tracheal wall Tr is formed by the expanded cuff 232 which is disposed in a subglottic region of the trachea Tr. Cuff 232 may be overinflated to ensure sealing using a pressure of at least 20 to 30 cm of water or higher. A syringe or pressure bulb may be coupled to port 220 to inflate the cuff 232. Cuff 232 may have any shape such as those previously described above including cylindrical, spherical, toroidal, oval, conical, etc. Cuff 230 is still in the unexpanded configuration. Other aspects of the endotracheal tube 200 are generally the same as described in FIG. 2. Other aspects of the anatomy are generally the same as described in FIG. 1. Optional mouth pad 210 is shown disposed over elongate shaft 212 but it may be omitted and added latter if desired or any mouth pad disclosed herein such as mouth pad 400 may be used.

FIG. 7E illustrates optional introduction of a fluid, antiseptic, or other therapeutic agent into port 218 as indicated by the arrow which then flows through its own discrete lumen and out fenestration 226 as indicated by the arrows. An example of an antiseptic may include chlorhexidine, and an example of another therapeutic agent may include an antibiotic. Examples of fluid may include water or saline. These examples of fluids may be used in this step of the procedure or any other fluid irrigation/lavage steps in this example of intubation and oral care illustrated in FIGS. 7A-7U. Fluid may be introduced into port 218 with a syringe, pump, or any other source of the fluid. The fluid fills the space superior to the inflated cuff 232 and is prevented from flowing down into the trachea due to the seal. Cuff 230 is still in the unexpanded configuration. This fluid irrigates or lavages the area and may have antiseptic agents that help minimize or eliminate microbial activity. Any volume of fluid may be used as needed such as for example 3 mL. Other aspects of the endotracheal tube 200 are generally the same as described in FIG. 2. Other aspects of the anatomy are generally the same as described in FIG. 1. Optional mouth pad 210 is shown disposed over elongate shaft 212 but it may be omitted and added latter if desired or any mouth pad disclosed herein such as mouth pad 400 may be used.

FIG. 7F shows that the fluid introduced via fenestration 226 in the previous step along with any debris from the area may then be removed from the same fenestration 226 as indicated by the arrows and suctioned out via port 218 as indicated by the arrow such by using a syringe coupled to port 218. In this step or any part of the procedure described here, low suction may be used in order to help prevent invagination of adjacent tissue into the fenestration which can block flow. The lower cuff is still in the expanded configuration and the upper cuff 230 is in the unexpanded configuration. Optional mouth pad 210 is shown coupled to elongate shaft 212 but may be omitted and added later, or any other mouth pad disclosed herein such as mouth pad 400 may be used. Other aspects of endotracheal tube 200 are generally the same as described in FIG. 2. Other aspects of the anatomy are generally the same as described previously in FIG. 1

FIG. 7G illustrates inflation of the upper cuff 230 while the lower cuff 232 remains inflated. Here, inflation fluid such as a gas like air or a fluid like water or saline may be introduced into port 208 as indicated by the arrow where the inflation fluid flows through its own lumen into upper expandable cuff 230 to inflate the cuff. The cuff, as previously discussed may have any shape including cylindrical, spherical, oval, conical, toroidal, etc. and when inflated forms a seal between the elongate shaft 212 and the wall of adjacent tissue. The cuff may be asymmetrically shaped with a thinner anterior portion that is configured to contact the back of the tongue T and a thicker posterior portion that is configured to contact the hypopharynx, pharynx or the region above the vocal chords in the larynx. The thicker and thinner sections allow the cuff to conform to the native anatomy.

Any pressure may be used but an example inflation pressure is between 20 to 30 cm of water. The cuff 230 is disposed in the pharynx or the hypopharynx or the region above the vocal chords in the larynx and may be inflated to any desired pressure. The upper cuff may push the epiglottis Ep out of the way. Optional mouth pad 210 is shown disposed on elongate shaft 212 but this may be coupled to shaft 212 later if desired, or any other mouth pad disclosed herein such as mouth pad 400 may be used. Other aspects of the endotracheal tube 200 are generally the same as described in FIG. 2. Other aspects of the anatomy are generally the same as described in FIG. 1.

FIG. 7H shows optional brushing with toothbrush Tb or swabbing with a swab Sw of the mouth M including the lips L, teeth To, tongue T and buccal pockets. A fluid such as water, saline, an antiseptic like chlorhexidine, or any other therapeutic agent may be used to swab and brush the oral cavity to reduce or eliminate pathogens as well as to loosen any unwanted debris in the oral cavity so that it may be removed from the mouth. The operator may also perform a visual inspection of the mouth to ensure that any lose debris has been removed. Both cuffs 230, 232 are in the expanded configuration. Other aspects of endotracheal tube 200 are generally the same as described in FIG. 2. Other aspects of the anatomy are generally the same as described previously in FIG. 1. If the optional mouth pad 210 or any other mouth pad disclosed herein such as mouth pad 400 is disposed on the elongate shaft 212 it may need to be moved proximally out of the way to provide access for the brush or swab to enter the mouth M. If the optional mouth pad is not coupled to the elongate shaft, then no repositioning of the mouth pad is needed.

FIG. 7I a syringe 704 or other irrigation or lavage device optionally may be inserted into the patient's mouth M to introduce fluid such as saline, water, an antiseptic, or other therapeutic agent into the oral cavity to rinse away the fluid and debris from the previous step. Because both cuffs 230, 232 are still in the expanded configuration, the fluid will not flow past the upper cuff 230 into the esophagus or trachea. The fluid may be suctioned out of the mouth with a suction wand, syringe, or it may be removed via fenestration 222 and port 206. If the optional mouth pad 210 is disposed on the elongate shaft 212 as seen here, it may need to be moved to allow the tip of the syringe or other device to be introduced into the mouth. If mouth pad 400 is used, then the fluid may be delivered with the ports in the mouth pad thereby eliminating the need to move the mouth pad away from the mouth. If the optional mouth pad has not yet been coupled to elongate shaft 212 then repositioning of mouth pad 210 is not needed. Other aspects of the endotracheal tube 200 are generally the same as described in FIG. 2. Other aspects of the anatomy are generally the same as described in FIG. 1.

FIG. 7J illustrates the optional mouth pad 210 optionally disposed over the elongate shaft 212. Any mouth pad disclosed herein such as mouth pad 400 may be used. The mouth pad may include a slotted region that allows the elongate shaft to slide through a wall of the pad into the central channel of the pad as previously described in FIGS. 3 and 4A-4B. The mouth pad optionally can then be slidably advanced distally until it apposes the lips L to close the patient's mouth and form a seal that prevents fluids from exiting the mouth M. The operator may need to hold the endotracheal tube to keep it stationary. The mouth pad may be soft and resilient to conform to the contours of the lips to form the seal. Other aspects of the endotracheal tube 200 are generally the same as described in FIG. 2. Other aspects of the anatomy are generally the same as described in FIG. 1. Both cuffs 230, 232 are still in the expanded configuration.

FIG. 7K shows an optional step of using an irrigation or lavage device 706 to introduce fluid such as saline, water, an antiseptic like chorhexidine or another therapeutic agent through the nares of the nose N into the sinus cavity Si. Any of the fluids used in this step or any other step in the method of FIGS. 7A-7U may be warmed, cold, or room temperature. Any volume of fluid may be used, for example 3-10 mL of saline. One nostril may be irrigated or lavaged at a time while the opposite nostril is pinched off to prevent fluid from flowing out of that nostril. The fluid then flows out of the sinus cavity Si into the pharynx or the region above the vocal chords in the larynx and then expanded cuff 230 prevents the fluid from flowing further downstream.

The fluid is then suctioned out of the pharynx or hypopharynx or the region above the vocal chords in the larynx, via fenestration 222 until mucous, secretions, and the fluid are thoroughly removed. Mouth pad 210 prevents the fluid from exiting the mouth. Any mouth pad such as mouth pad 210 or 400 may be used. The fluid cleans and may have antiseptic affect in the nasal, sinus, pharynx, or the region above the vocal chords in the larynx, as well as removing any loose and unwanted debris from those areas. The fluid or debris may be removed via a syringe or other vacuum device coupled to port 206 as shown by the arrow. Alternatively, the fluid and debris may also be suctioned out of the patient's mouth with a suction wand. Other aspects of the endotracheal tube 200 are generally the same as described in FIG. 2. Other aspects of the anatomy are generally the same as described in FIG. 1. Both cuffs 230, 232 are still in the expanded configuration.

FIG. 7L illustrates an optional step of cleaning or sanitizing the oral cavity. Here, fluid such as saline, water, antiseptic such as chlorhexidine, or another therapeutic agent is introduced into port 206 such as with a syringe, or a pump, or other source of the fluid. For example, 10-30 mL of chlorhexidine may be used. Another example of a fluid that may be introduced in this step or any of the fluid introduction steps in FIGS. 7A-7U is a lubricant, oral antibiotic, or other sterile vehicle. The fluid then flows along an independent lumen and exits fenestration 222 just superior to cuff 230 as shown by the arrows. The fluid then fills the pharynx or the region above the vocal chords in the larynx and the oral cavity of the mouth M. The fluid cleans and may reduce or eliminate pathogens in the oral cavity as well as remove loose debris. The fluid may then be removed from the oral cavity by suctioning it out of fenestration 222 and out port 206 in the opposite direction that it was introduced. Or, the fluid may be removed from the oral cavity by suctioning it out of the mouth with a suction wand or other device that is inserted past the mouth pad 210 and through the lips L into the mouth M. Any mouth pad disclosed herein such as mouth pad 400 may be used. Other aspects of the endotracheal tube 200 are generally the same as described in FIG. 2. Other aspects of the anatomy are generally the same as described in FIG. 1. Both cuffs 230, 232 are still in the expanded configuration.

FIG. 7M shows an alternative way of cleaning and sanitizing the oral cavity that may be used alone or in conjunction with the technique described above in FIG. 7L. Here, a syringe or other irrigation/lavage device 708 is inserted past the mouth pad 210 if present into the mouth M and fluid is introduced from the syringe into the mouth M. Any mouth pad disclosed herein may be used including mouth pad 400. If mouth pad 400 is used, the fluid may be introduced via ports 402, 404 and the mouth pad 400 may remain apposed with the patient's lips and mouth to maintain a seal. The fluid may be any of those described with respect to FIG. 7L above such as water, saline, an antiseptic, or other therapeutic agent that can be used to cleanse and sanitize the oral cavity. The fluid may be removed from the oral cavity by suctioning it out with the syringe used to introduce the fluid, or by using a suction wand or other suction device. The fluid may also be removed from the oral cavity by suctioning it out of fenestration 222 and out port 208. Other aspects of the endotracheal tube 200 are generally the same as described in FIG. 2. Other aspects of the anatomy are generally the same as described in FIG. 1. Both cuffs 230, 232 are still in the expanded configuration.

FIG. 7N illustrates removal of the fluid from the oral cavity after it has been introduced via fenestration 222 in FIG. 7L and/or via syringe 708 in FIG. 7M or the ports in the mouth pad 400. Here, the fluid is removed via fenestration 222 and collected at port 206 by a syringe, a pump, or other collection device. Optionally or in conjunction with this fluid removal technique, the fluid may also be removed via the mouth using a syringe or suction wand, as previously disclosed. Other aspects of the endotracheal tube 200 are generally the same as described in FIG. 2. Other aspects of the anatomy are generally the same as described in FIG. 1. Both cuffs 230, 232 are still in the expanded configuration.

After the fluid and debris have been removed from the oral cavity, the mouth pad 210 or any mouth pad disclosed herein such as mouth pad 400 is removed from the elongate shaft 212 so the mouth is no longer sealed. FIG. 7O shows the endotracheal tube 200 disposed in the patient's throat with both the upper and lower cuffs 230, 232 inflated and the mouth pad removed. Other aspects of the endotracheal tube 200 are generally the same as described in FIG. 2. Other aspects of the anatomy are generally the same as described in FIG. 1.

FIG. 7P shows an optional additional suctioning of any remaining fluid or debris in the oral cavity using suction wand 710. The suction wand such as a Yankauer device may be moved into different positions in the oral cavity to ensure that fluid and debris are thoroughly removed. Other aspects of the endotracheal tube 200 are generally the same as described in FIG. 2. Other aspects of the anatomy are generally the same as described in FIG. 1. Both cuffs 230, 232 are still in the expanded configuration.

FIG. 7Q shows deflation of the upper cuff 230 after the oral hygiene has been performed. Here inflation fluid is removed from cuff 230 through its own independent lumen via port 208 as indicated by the arrow. Other aspects of the endotracheal tube 200 are generally the same as described in FIG. 2. Other aspects of the anatomy are generally the same as described in FIG. 1. While cuff 230 is deflated, cuff 232 remains in the expanded configuration.

FIG. 7R shows an additional optional irrigation or lavage step after the upper cuff 230 has been deflated. Here, fluid such as saline, water, antiseptic such as chlorhexidine or another therapeutic agent is introduced into port 218 (as indicated by the arrow) with a syringe, pump, or other source of the fluid which then flows along an independent lumen and exits the elongate shaft at fenestration 226 which is superior to the lower cuff 232 which is still in the expanded configuration. The fluid helps reduce or eliminate pathogens and debris from the subglottic part of the trachea Tr that is superior to the expanded cuff 232. Any volume of fluid may be used such as 3 mL of saline. Once the irrigation/lavage is complete the fluid may be removed. Other aspects of the endotracheal tube 200 are generally the same as described in FIG. 2. Other aspects of the anatomy are generally the same as described in FIG. 1. Cuff 230 is deflated while cuff 232 is still inflated.

FIG. 7S shows removal of the fluid introduced in FIG. 7R by suctioning the fluid or debris out of fenestration 226, through an independent lumen and out port 218 (as indicated by the arrow) into a syringe, or other collection device. Other aspects of the endotracheal tube 200 are generally the same as described in FIG. 2. Other aspects of the anatomy are generally the same as described in FIG. 1. Cuff 230 is deflated while cuff 232 is still in the expanded configuration. Of course, one of skill in the art will appreciate that if the irrigation/lavage in FIG. 7R is not performed then the suctioning in FIG. 7S need not be performed.

FIG. 7T shows optional partial deflation of the lower cuff 232. When cuff 232 is initially inflated, it may be overinflated in order to ensure a tight seal so that fluids do not pass the cuff. However, over inflation can cause tissue trauma and therefore is not recommended for long periods of time. Therefore, if the cuff 232 was initially overinflated then optionally it may also be partially deflated so that it still engages tissue and forms a seal without fear of causing tissue trauma. Thus, fluid is removed from cuff 232 along its independent lumen and then removed via port 220 (as shown by the arrow) with a syringe or other fluid collection device. Any pressure may be used in the cuff but in an example the pressure is reduced to about 20 to 30 cm of water. Other aspects of the endotracheal tube 200 are generally the same as described in FIG. 2. Other aspects of the anatomy are generally the same as described in FIG. 1. Cuff 230 is deflated while cuff 232 is still in the expanded configuration but not necessarily overinflated.

After the endotracheal tube has been inserted into the trachea and the oral care has been administered, the connector 216 on the proximal end of the endotracheal tube may be coupled to tubing (not shown) which is attached to a ventilator (also not shown). The ventilator mechanically delivers air to and from the patient's lungs. The oral and nasal cleaning procedure time should be minimized as much as possible. For example, the procedure time to perform oral and nasal cleaning after intubation should be less than 5, 4, 3, 2, or 1 minute(s).

Optionally, the endotracheal tube may be used to help manage nasal and oral secretions in patents that demonstrate need by leaving the oropharyngeal cuff inflated (upper cuff), the cuff may also be a cuff disposed in the region above the vocal chords in the larynx. And any secretions collected above the upper cuff may be removed via fenestration 222.

FIG. 7U shows an optional step of inserting an orogastric feeding tube 712 into fenestration 234. The orogastric tube then passes through an independent lumen until the orogastric tube exits fenestration 224 which is inferior to cuff 230. The orogastric tube is then advanced into the esophagus Es and toward the stomach for feeding purposes. Other aspects of the endotracheal tube 200 are generally the same as described in FIG. 2. Other aspects of the anatomy are generally the same as described in FIG. 1. Cuff 230 is deflated while cuff 232 is still in the expanded configuration. In the case where an orogastric tube is used, the endotracheal tube may have a lumen configuration such as seen in FIG. 5 above. If no orogastric feeding tube is used then the lumen configuration may be that seen in FIG. 6 and one of skill in the art will appreciate that fenestrations 224, 234 seen in FIGS. 7A-7U may be omitted.

Any or all of the cleaning and sanitizing steps of the method described above in FIGS. 7A-7U may be used or omitted and may be performed in any desired order. Additionally any or all of the steps may be repeated from time to time or with regular frequency and in any order while the patient is intubated, to ensure that the patient's endotracheal tube and adjacent tissue remains clean, free of biofilm, and with none or a reduce amount of pathogens to minimize the chances of causing ventilator acquired pneumonia (VAP).

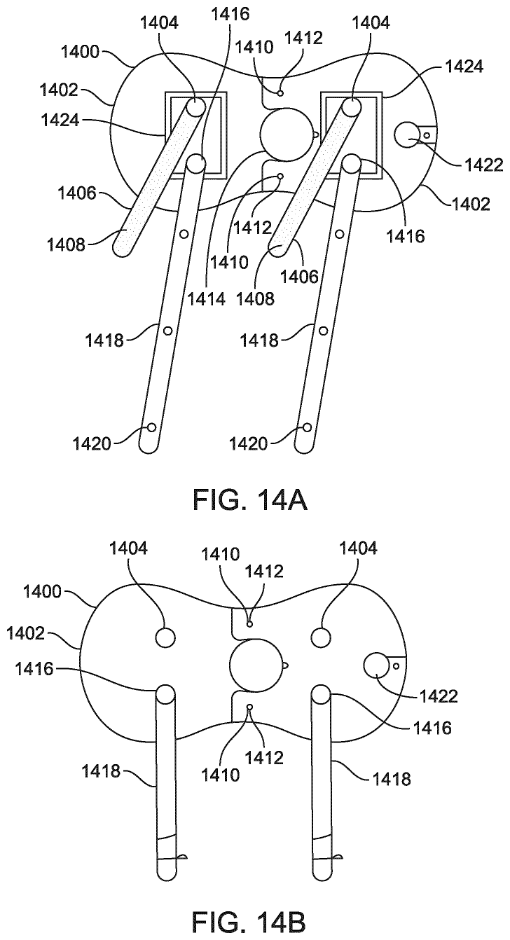

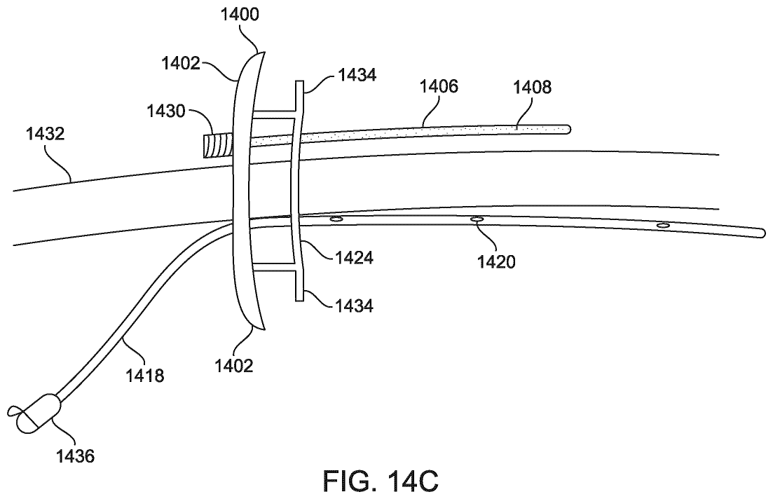

FIGS. 14A-14C show another example of a mouth pad 1400 (also referred to as a mouthpiece) that may be used with any of the endotracheal tubes disclosed herein or with any standard commercially available endotracheal tube.

FIG. 14A shows a back side of a mouth pad 1400 (the side that apposes the patient's mouth). The mouth pad 1400 may be made of any of the materials discussed herein including a resilient material that will conform with and help seal against the patient's mouth to prevent aerosols, droplets or other fluids from escaping the patient's mouth, and may be a single integral mouth pad or may include two or more portions 1402 (e.g. left and right, or first and second portions) that may be separate and discrete from one another and then may be joined using any of the joining techniques described herein or otherwise known in the art. For example, one half may have a U-shaped end and include a pin 1410 or other protuberance on the legs of the U that can be inserted or snapped into an aperture 1412 in the legs of the other U-shaped half. In this example, there are two pins, one on each leg on the half portion of the mouth pad that snap into cooperating apertures on the legs of the other U shaped half of the mouth pad to join the two mouth pad halves together and to form a central aperture 1414 through which any endotracheal tube may be placed. Any of the other coupling techniques disclosed herein may be used in combination with or substituted for the pins and apertures of FIG. 14A. Each half 1402 of the mouth pad may include an aperture 1404 for an irrigation tube 1406 with a plurality of irrigation fenestrations 1408 that may extend into the mouth and optionally into the upper throat area. The irrigation tube 1406 may be integral with the mouth pad or it may be inserted through the aperture 1414 and extend inward into the patient's mouth with a portion extending away from the mouth pad on the opposite side to allow it to be coupled to a fluid source (which may be any of the irrigation fluids disclosed herein) to irrigate, clean or sanitize the patient's mouth, and portions of the upper throat.

Each half of the mouth pad may also include an aperture 1416 for a suction line 1418 that may include one or more suction fenestrations 1420. The suction line 1418 may be integral with the mouth pad or coupled to the mouth pad. The suction line may be coupled to a vacuum source such as a hospital suction line (e.g. wall mounted hospital vacuum source) and extend inward into the patient's mouth and optionally into a part of the throat. The suction may be used to remove the irrigation fluid introduced into the patient's mouth with the irrigation lines 1406.

One or both halves of the mouth pad may include another aperture 1422 for an orogastric tube whose use and placement is generally the same as previously described in this specification.

Optionally, one or both halves of the mouth pad may also include a line protector 1424 which extends away from the back surface of the mouth pad towards the patient's mouth, The line protector 1424 may be a wall that either forms an enclosed region or may be an open region. In this example, the wall forms a closed rectangular region although this is not limiting and the wall may form any shape such as an open or closed circle, square, rectangle, ellipse, ovoid, triangle, or any other desired shape. The wall extends outwardly and engages the lips and extends into the patient's mouth. The line protector may be disposed between the patient's upper and lower front teeth and this prevents the teeth from damaging the lines. The line proctor may be rigid or resilient and may act as a bite block. The line protector may also include an optional flange to fit behind the patient's teeth to help align and secure the mouth pad into a desired position as will be discussed below.

FIG. 14B shows the opposite side of the mouth pad 1400, here the top side or front side of the mouth pad that faces away from the patient's mouth. The through hole 1404 for the irrigation line may have irrigation tubing extending away from the aperture and may be coupled to a syringe or other irrigation line for providing any of the irrigation fluids disclosed herein. A Luer connector may be coupled to mouth pad aperture 1404 to allow a syringe or other tubing to be easily coupled or uncoupled from the mouth pad.

Similarly, suction line 1418 may extend through aperture 1416 and be coupled to a vacuum source such as a hospital's wall mounted suction. The end of the suction line 1418 may include a connector to allow it to be easily coupled or uncoupled to another tubing line. Other aspects of FIG. 14B have been described with respect to FIG. 14A.

FIG. 14C shows a side view of the mouth pad in FIGS. 14A-14B. Here, the line protector 1424 includes an optional flange 1434 that extends upward and downward from the line protector 1424 and is configured to fit behind the patient's teeth to help align and secure the mouth pad into a desired position. The flange may be a rigid or resilient material such as a polymer. The irrigation tube 1406 with irrigation fenestration 1408 extends away from the mouth pad 1400 into the patient's mouth for irrigation of the mouth and upper throat. A connector 1430 such as a Luer connector is coupled to the front of the mouth pad to allow the irrigation line to be fluidly coupled to a syringe or other irrigation line, or the line may extend out far enough to be coupled directly to the irrigation fluid source with or without a connector. The suction line 1418 with suction fenestrations 1420 also extends away from the mouth pad into the patient's mouth for suction of the mouth and upper throat region. The suction line 1418 extends through the mouth pad 1402 and has a free end with a connector 1436 that may be connected to a source of vacuum such as wall mounted hospital suction, or connected to another suction line, or the suction line may be terminated adjacent to the mouth pad and be coupled to another suction line that is coupled to the vacuum source.

FIG. 14C also shows that any endotracheal tube may be used with the mouth pad of FIGS. 14A-14C. Therefore any of the endotracheal tubes disclosed herein may be used with the mouth pad of FIGS. 14A-14C or a standard commercially available endotracheal tube may be used instead. If an endotracheal tube disclosed herein is used with the mouth pad of FIGS. 14A-14C, then any of the optional features disclosed herein may also be used in combination with or substituted with any of the features described with respect to FIGS. 14A-14C. Additionally, the method of use will be generally the same as the methods described previously.

If a standard endotracheal tube is used, then the standard endotracheal tube typically has only a single subglottic inflation cuff for sealing the endotracheal tube in the patient's throat. Thus, irrigation, cleaning and sanitization of the patient is limited to the mouth and throat region above the subglottic cuff, and similarly suctioning is also limited to this region. One of skill in the art will therefore appreciate that the methods described herein may be modified to accommodate a standard endotracheal tube such as inflation of only a single cuff and irrigation/suctioning of the region above the cuff but otherwise are generally the same as previously described.

The external connections for either the suction and/or irrigation lines as well as the internal suction and irrigation lines for the example in FIGS. 14A-14C or any other example disclosed herein may optionally be replaced as needed such as due to damage, fouling, contamination, or any other reason. The internal lines (the lines that extend into the patient's mouth or throat) may be removed from the patient and uncoupled from the mouth pad. They may then be replaced with new lines. Similarly, the external lines may be disconnected from the mouth pad and replaced with new lines as required.

Also, in the example of FIGS. 14A-14C as well as any other example disclosed herein, suction and irrigation may be applied individually or concurrently.

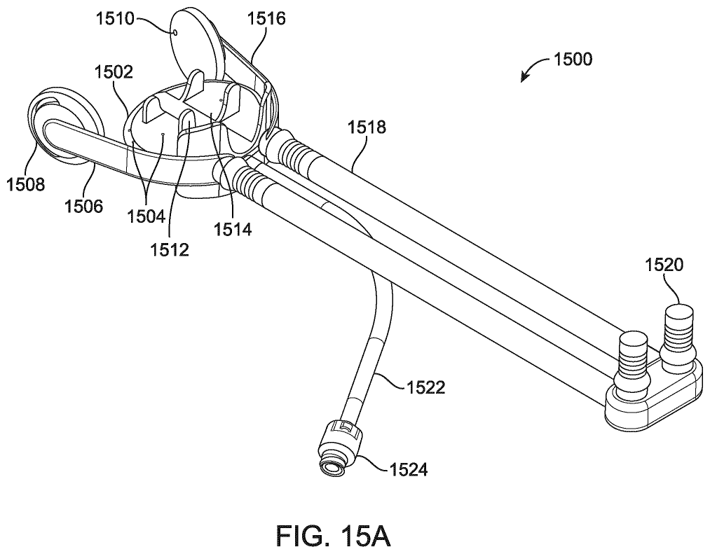

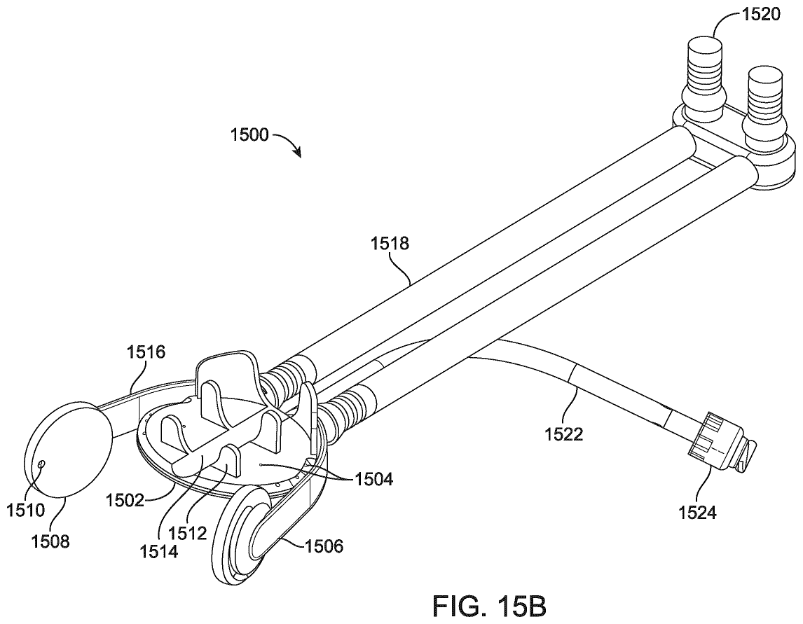

FIGS. 15A-17B illustrate another example of an irrigation device that may be coupled to a standard endotracheal tube and used to irrigate (or lavage or instill) an oral cavity in a ventilated patient to help cleanse the oral cavity and then suction out the irrigation fluids.

FIG. 15A shows a perspective view of irrigation and suction device 1500 which includes an optional irrigation plate 1502 which may be any shape but in this example is generally a circular disc shape. FIGS. 15A-21 describe examples of the device with an irrigation plate, but one of skill in the art will appreciate that this optional element may be removed from the device and the irrigation outlets may be relocated onto the suction element or anywhere else on the device as desired. Similarly the irrigation line and cradle may also be relocated onto the suction element if the irrigation plate is omitted. One or more irrigation outlets 1504 are disposed in the irrigation plate 1504. The irrigation outlets may be located anywhere on the irrigation plate, but in this example the irrigation outlets 1504 may be disposed on an upper surface of the irrigation plate, or a lower surface of the irrigation plate, or along a perimeter of the irrigation plate. In this example, irrigation outlets are located in all three positions. The irrigation outlets allow fluid to exit from the irrigation plate to irrigate an oral cavity in the patient such as the mouth and portions of the back of the throat. The fluid used to irrigate the oral cavity may be water, saline, a therapeutic fluid such as an antiseptic used to kill or reduce unwanted bacteria or other microorganisms in the patient's oral cavity, an antibiotic, or a lubricant. Any of the fluids described in this specification may be used with this device. A reservoir may be included in the irrigation plate (not shown) that can hold and help disburse irrigation fluid to the irrigation outlets. A flexible fluid line 1522 may be fluidly coupled to the irrigation plate 1502 to provide irrigation fluid to the device. A connector 1524 such as a Luer connector, a barbed fitting, or other connector known in the art may be coupled to the proximal end of the flexible fluid line 1522 in order to facilitate connection of the fluid line with a fluid source that can deliver the fluid to the device.

An optional cradle may be coupled to the irrigation plate. In this example the cradle is formed from several protrusions 1512 that extend outward from a top surface of the irrigation plate. Adjacent protrusions 1512 are separated by a gap 1514 and the gap is sized and shaped to receive an endotracheal tube. Here the gap is semi-circular in shape and thus each protrusion forming the gap has a quarter circle shape on a side facing the gap. Also the upper surface of the irrigation plate may have a concave channel that is also sized and shaped to receive an endotracheal tube. In this example, the cradle has four protrusions with each pair forming a gap for receiving the endotracheal tube thereby providing two regions of contact between the cradle and the endotracheal tube. The gap may be tight enough to provide a friction fit to hold the endotracheal tube, or the cradle may simply loosely hold the endotracheal tube. In examples where there is no irrigation plate, the optional cradle may be coupled to the suction element which is described below.

A suction element 1506 may be coupled to the irrigation plate 1502 or may be alone when there is no irrigation plate. The suction element 1506 is used to suction out the fluid previously irrigated into the oral cavity. Here, the suction element includes two paddles each having an arm 1516 and an enlarged head 1508 coupled to a distal end of the arm. The paddles are coupled together at their proximal end to form a U-shaped or V-shaped suction element. The arms may have any cross-section. In this example, the cross-section of the arms is rectangular, although other shapes such as square, circular, elliptical, etc. are also contemplated. The arms may be stiff and rigid or they may be flexible to facilitated insertion into the patient's mouth. The enlarged head may also be any shape but in this example is a circular disc. Suction inlets 1510 may be disposed anywhere on the enlarged head or the arms of the suction element. Additional examples will be described below. A suction line 1518 may be fluidly coupled to each arm to provide vacuum to the suction inlets on the arm or enlarged head. In this example, two suction lines are included, one suction line coupled to each arm and enlarged head. Although the two suction lines can easily be replaced with a single suction line fluidly coupled to both arms and heads. The suction line may be rigid or flexible as desired. The proximal end of the suction line 1518 may include a connector 1520 to facilitate connection of the suction line with a vacuum source. In an alternative example, the suction element may only include a single paddle with an arm and enlarged head that fits in the buccal pocket between the check and teeth. In either example (one paddle or two paddles), the patient may be positioned on his or her lateral side to create a low point in the patient's oral cavity where the irrigation fluid can accumulate and then the enlarged head of the suction element can easily suction the irrigation fluid out of the low point. In this example, the enlarged head is positioned in the buccal pocket between the cheek and teeth, and therefore the low point is also in the buccal pocket.

In the example where the optional irrigation plate is omitted, then one of skill in the art will appreciate that the irrigation outlets will be incorporated onto portions of the suction element, and the irrigation line will also be fluidly coupled with the irrigation outlets on the suction element. Thus, irrigation outlets may be disposed along the arms or on the enlarged head region of the suction element and an irrigation line coupled thereto. Also, the optional cradle may be coupled to the suction element instead of the irrigation plate.

FIG. 15B is also a perspective view of irrigation and suction device 1500 in FIG. 15A but from a different angle and therefore it takes the same form as previously described with respect to FIG. 15A.

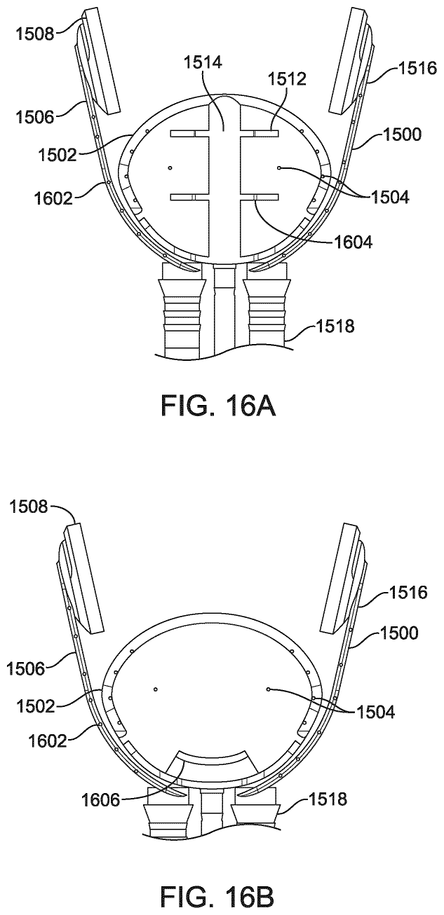

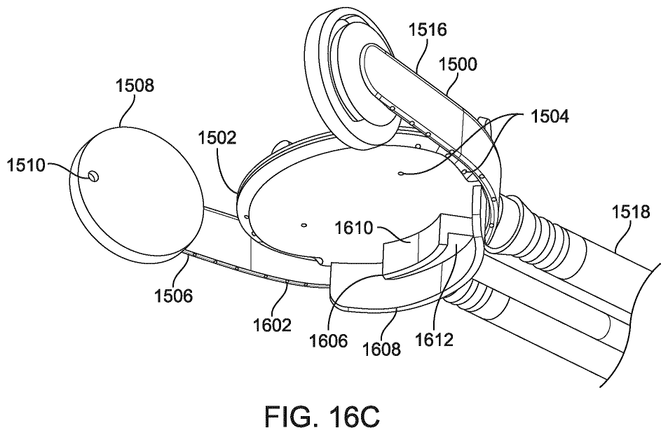

FIG. 16A shows a top side of the optional irrigation plate 1502 from FIGS. 15A-15B. In this view, the gap 1514 is more clearly visible between protrusions 1512. Here, the gap also has a concave channel in the upper surface of the irrigation plate to receive the endotracheal tube. Optionally, irrigation outlets 1602 may be included on the upper surface of the arms 1516 or on the lower surface or inner side or outer side of one or both arms 1516. One or any number of irrigation outlets may be included on any of these surfaces of the arms in order to provide a desired irrigation pattern to the oral cavity being irrigated. The upper surface also may have any number or pattern of irrigation outlets 1504 on the upper surface of the irrigation plate. The protrusions 1512 also may have optional irrigation outlets 1604 disposed thereon. The lateral sides of the arms may also have irrigation outlets (not shown). Other aspects of FIG. 16A are generally the same as previously described above in FIGS. 15A-15B.

FIG. 16B shows a bottom view of the optional irrigation plate 1502 seen in FIG. 16A. Again, optionally irrigation outlets may be included on bottom surface of the irrigation plate 1502, around the perimeter of the irrigation plate, on the top, bottom, inner side or outer side surfaces of arms 1516 or anywhere else on the device if desired. Any example may optionally include a teeth registration element 1606, here on the bottom surface of the irrigation plate, although any surface may also have a teeth registration element. The teeth registration element will be illustrated and described more clearly below in FIG. 16C. Other aspects of FIG. 16B are generally the same as previously described above in FIG. 16A.

FIG. 16C is a perspective view of the bottom of the optional irrigation plate 1502 discussed above in FIGS. 16A-16B. The teeth registration element 1606 is more clearly visible in this view and includes anterior wall 1608, and posterior wall 1606 couped to the irrigation plate 1502 and extending downward and away from the irrigation plate. A gap 1612 is disposed between the anterior and posterior walls 1608, 1606. The gap 1612 is sized to receive a patient's lower front teeth and the teeth registration element 1610 helps align the irrigation device in the patient's mouth and prevents unwanted movement. Other aspects of FIG. 16C are generally the same as previously described above in FIGS. 16A-16B. When the optional irrigation plate is omitted from the device, then the irrigation outlets may be disposed anywhere along the suction element as described above and the irrigation line coupled to the irrigation outlets. The teeth registration element and cradle are optionally coupled to the suction element.

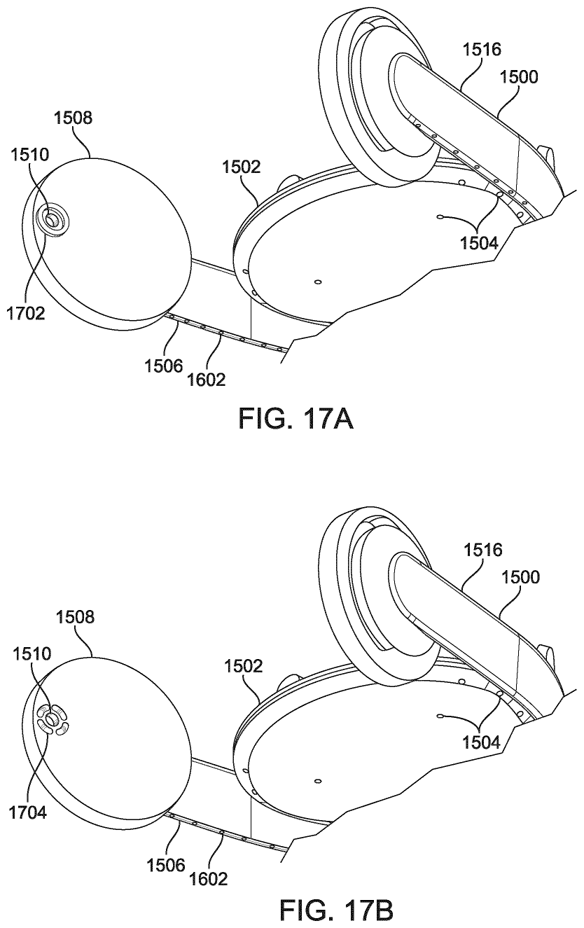

FIG. 17A shows a perspective view of the distal end of the irrigation device 1500 such as previously described above but with an optional suction guard 1702 adjacent a suction inlet 1510. The suction guard 1702 is a wall that protrudes away from the enlarged head 1508 and prevents tissue from being sucked into the suction inlet 1510. The suction guard may have any shape but in this example is an annular ring disposed around the suction inlet 1510. The suction guard may be used in conjunction with any other suction inlet on the device and is not limited to being disposed on the enlarged head. Therefore, suction guards may be adjacent suction inlets on the arms or irrigation plate, or wherever a suction inlet may be located. Other aspects of the irrigation device 1500 are generally the same as previously described above and are not repeated here for the sake of brevity.

FIG. 17B shows still another example of a suction guard 1704 that may be used with any of the irrigation devices disclosed herein. Here, the suction guard 1704 is disposed adjacent a suction inlet 1510 disposed on the enlarged head 1508. The suction guard 1704 in this example is an annular wall that protrudes away from the surface of the enlarged head 1508. The wall is discontinuous and comprises a plurality of arcuate segments that are adjacent one another to form a broken ring. The suction guard prevents tissue from being sucked into the suction inlet, and the gaps in the wall of the suction guard still allow effective suction to be provided. The suction guard may be used with any suction inlet on the irrigation device 1500 including but not limited to suction inlets on the enlarged head 1508, the arms or irrigation plate. Other aspects of the irrigation device 1500 in FIG. 17B are generally the same as previously described above and are not repeated for the sake of brevity.

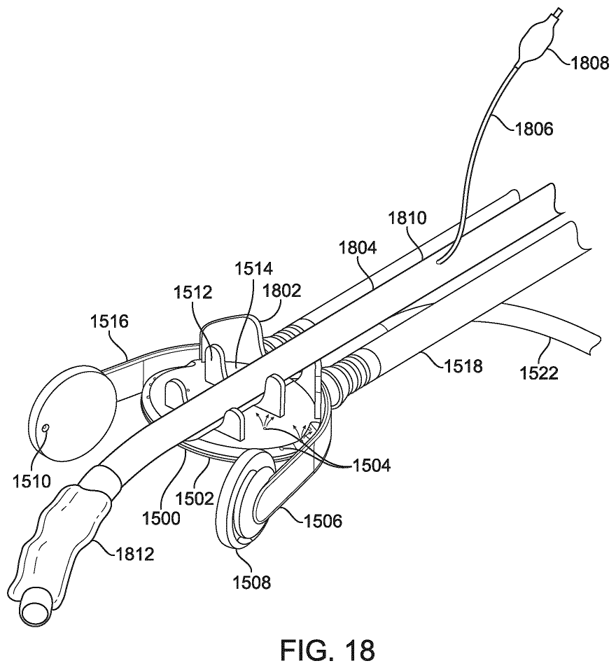

FIG. 18 shows an irrigation device 1500 coupled to an endotracheal tube 1804. Here, the irrigation device 1500 may be any of the irrigation devices disclosed herein, but in this example is the irrigation device 1500 described above in FIGS. 15A-15B, and therefore generally takes the same form as described in those figures and the detailed description will not be repeated for the sake of brevity. The endotracheal tube is any standard endotracheal tube 1804 that may be used in a patient on a ventilator. The endotracheal tube 1804 includes an elongate tube 1810 that has a proximal end and a distal end. The distal end is inserted into the patient's trachea during intubation and a balloon or inflation cuff 1812 is inflated to seal the passage and also anchor the endotracheal tube in a desired position. The proximal end extends out of the patient's mouth and the proximal-most end (not shown) may be coupled directly or indirectly to a ventilator. A lumen in the endotracheal tube is fluidly coupled to the balloon 1812 and an extension line 1806 is also coupled to the lumen. A connector such as a Luer or barb fitting 1808 allows the line to be coupled to a syringe or other inflation device to inflate or deflate the balloon as desired. A mouthpiece (also referred to as mouth pad) such as any of those previously described above may be coupled to the endotracheal tube either before, during or after the endotracheal tube is inserted into the patient in order to prevent fluids, droplets or aerosols from exiting the patient's oral cavity. One of skill in the art will appreciate that the mouthpieces disclosed herein may require some modification in order to accommodate the different geometry of the examples illustrated in FIGS. 15A-21.

The endotracheal tube 1804 is placed in the cradle formed by the protrusions 1512 which are separated by gap 1514. Here the cradle has two rows of protrusions so the cradle has two regions of supporting the endotracheal tube. A proximal wall 1802 with an arcuate cutout also is sized and shaped to receive the endotracheal tube to provide another region for supporting and engaging the endotracheal tube with the irrigation device. The elongate shaft of the endotracheal tube runs substantially parallel with the longitudinal axis of the irrigation device. The elongate shaft of the endotracheal tube may run on top of the suction lines 1518. In some cases, the irrigation device 1500 may have two suction lines that are substantially parallel with one another with a small gap therebetween. The elongate shaft of the endotracheal tube may sit in the gap and be supported by the two suction lines. In use, fluid may flow in a spray or jet out of the irrigation outlets, as indicated by the arrows. As previously discussed, the irrigation outlets may be on the top, bottom or perimeter of the irrigation plate or anywhere on the device including the top, bottom, inner side or outer side of the arms or on the enlarged head. In the example where there is no cradle, the irrigation device may simply rest under or over the endotracheal tube.

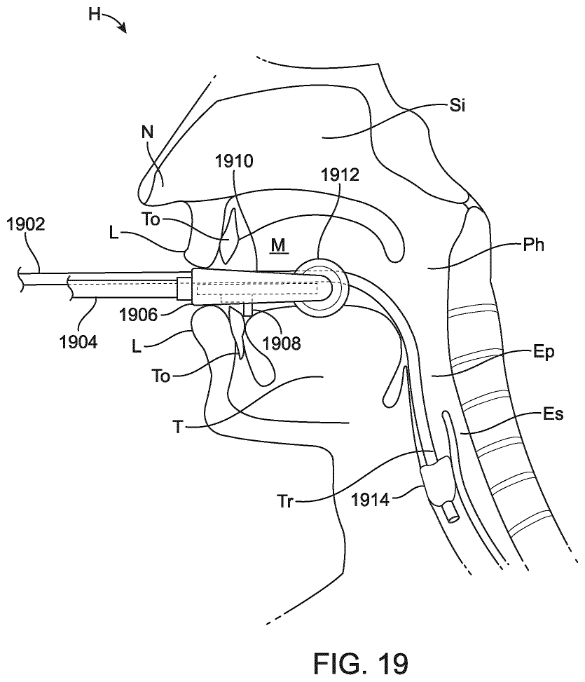

FIG. 19 shows a method of use of any of the irrigation devices disclosed herein coupled with a standard endotracheal tube to treat a patient. Here, the irrigation device 1904 is substantially the same as the irrigation device 1500 described in FIGS. 15A-15B above, and the endotracheal tube 1902 is substantially the same as the endotracheal tube in FIG. 18. In use, the patient is intubated using a standard endotracheal tube and the distal end of the endotracheal tube is disposed in the tracheal and the distal balloon or cuff 1914 is inflated to seal the trachea and anchor the endotracheal tube into position. The irrigation device 1904 is then coupled to the endotracheal tube by seating the endotracheal tube into the cradle on the irrigation device as previously described in FIG. 18. If the optional cradle is not used, then the irrigation device may simply lie over or under the endotracheal tube. The optional irrigation plate is disposed in the oral cavity and the enlarged head 1912 of the suction element is disposed in the oral cavity, such as between the cheek and the teeth in the buccal pocket. The arms 1910 of the suction element may also be disposed in the space between the cheek and teeth. The irrigation device is inserted into the oral cavity until the lower teeth To engage the teeth registration element 1908. The proximal end of the suction lines, irrigation lines and endotracheal tube extend out of the patient's mouth. The device may then be used to irrigate the oral cavity and suction out the irrigation fluids, thereby cleaning and disinfecting the oral cavity. The fluid may be water, saline, or any other fluid such the antiseptics, antibiotics or lubricants disclosed herein. Additional cleaning steps such as brushing the teeth or oral cavity with a brush or any of the other cleaning steps previously described above may also be used in conjunction with this device and method of use. The subglottic region (e.g. below the glottis and above the inflatable cuff) may be irrigated and suctioned separately using a separate irrigation and suction line if desired. Other aspects of the anatomy in FIG. 19 are generally the same as previously described in FIG. 1. Additionally, any of the mouth pieces described here may be used with the irrigation device 1500 to prevent splashing out of the mouth. One of skill in the art will appreciate that the mouthpiece may require modification in order to fit with the tubing arrangement of the irrigation device. Also one of skill in the art will appreciate that the irrigation and suctioning not only cleanses or sanitizes the oral cavity but similarly cleanses or sanitizes portions of the endotracheal tube.

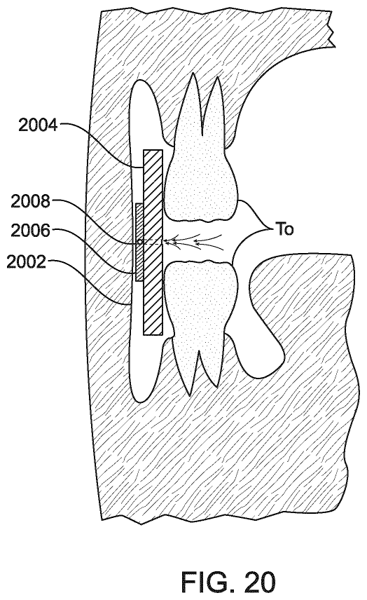

FIG. 20 shows a cross-section of the patient's mouth in order to more clearly illustrate positioning of the suction element in the buccal pocket that exits between the cheek 2002 and teeth, To as discussed in FIG. 19. Here, both the enlarged head 2004 and the arm 2006 of the suction element are disposed in the gap between the cheek 2002 and teeth To. Suction inlet 2008 suctions out fluid in the oral cavity. Thus, as irrigation fluid pools in the mouth, it may be suctioned out of the oral cavity using the suction elements on the device and as illustrated by the arrows. In the example where the optional irrigation plate is omitted, then the irrigation outlets would also be disposed on the enlarged head or arms of the suction element and therefore they too may also be disposed in the buccal pocket.

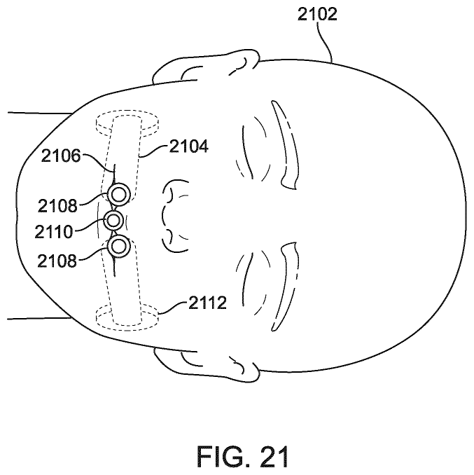

Because the suction element is disposed in the gap between the cheek and the teeth as shown in FIG. 20, it may be advantageous to lay the patient on a lateral side so that irrigation fluid accumulates at the low point. FIG. 21 shows the patient 2102 laying on a lateral side so that at least one of the enlarged heads 2112 connected to an arm 2104 of the suction element is at a low point in the oral cavity where irrigation fluid will accumulate and can then be easily suctioned out by the suction element. The patient may be turned onto the opposite lateral side to allow fluid to accumulate and be suctioned from that side if desired. The endotracheal tube 2110 and the two suction lines 2108 extend out of the patient's mouth 2106. The irrigation line is not shown in this figure for simplicity. The irrigation device may be used to simultaneously irrigate and suction fluids continuously if needed. Suction may be applied to only one arm of the suction element when the patient is on his/her lateral side rather than applying suction to both arms. This ensures suction is applied to the lowest point in the oral cavity.

Any of the examples of endotracheal tubes, systems or methods disclosed herein provide mechanical blockage, disinfection, as well as large volume irrigation/suctioning of the nasal and oral cavities while minimizing or eliminating the risk of translocation of harmful bacteria and viruses to the subglottic region.

Optionally, any of the suctioning steps may be performed using high pressure suctioning (e.g. high negative pressure, or high vacuum) which may use up to 150 mm Hg of vacuum applied without causing tissue to be drawn into fenestrations and causing obstruction.

Optionally, any of the irrigation steps may be performed using high pressure irrigation which may use 3.5 mL of fluid per second or greater.

Optionally, any of the lavage steps may use high volume flow rates which theoretically are unbounded and can be any flow rate since fluid is suctioned and removed as quickly as it is introduced through the nares and mouth via the fenestrations in the endotracheal tube. Lumen size and fenestration sizes may be adjusted to accommodate differing flow rates.

In addition to helping maintain oral hygiene the device and method disclosed herein may provide additional protection against other pathogens such as COVID 19 virus by preventing or minimizing generation of aerosols which may contain the virus or other pathogens. Use of the mouth pad and irrigation/lavage and sanitization procedures disclosed herein help avoid open airway suctioning as well as minimize or prevent aerosolization of pathogens. This is because an open airway is eliminated by use of the mouth pad and the increased irrigation/lavage and sanitization of the oral cavity, thereby reducing aerosols and providing additional protection to front line critical care staff. Optionally, any example of endotracheal tube disclosed herein may be coated or otherwise incorporate the use of antimicrobials to help reduce or eliminate pathogens. For example, silver may be applied to the endotracheal tube, or other antimicrobial coatings may be applied to the endotracheal tube to help kill pathogens.

### Notes and Examples

The following, non-limiting examples, detail certain aspects of the present subject matter to solve the challenges and provide the benefits discussed herein, among others.

Example 1 is an endotracheal tube configured for insertion into a trachea of a patient, said endotracheal tube comprising: an elongate shaft having a proximal end, a distal end, a proximal opening at the proximal end, a distal opening at the distal end, and a tracheal lumen extending between the proximal and distal openings, the tracheal lumen configured to deliver oxygen to a patient's lungs; a lower sealing cuff disposed adjacent the distal end of the elongate shaft, the lower sealing cuff having an expanded configuration and a collapsed configuration, wherein the lower sealing cuff is configured to be disposed in the trachea, and wherein in the expanded configuration the lower sealing cuff is configured to seal the trachea against the elongate shaft; a lower sealing cuff lumen fluidly coupled with the expandable lower sealing cuff and extending proximally toward the proximal end of the elongate shaft, the lower sealing cuff lumen configured to deliver an inflation fluid to and from the lower sealing cuff for inflation or deflation thereof; a lower fenestration in a wall of the elongate shaft, the lower fenestration disposed adjacent the lower sealing cuff and superior thereto; a lower fenestration lumen fluidly coupled with the lower fenestration and extending proximally therefrom toward the proximal end of the elongate shaft, the lower fenestration lumen configured to deliver fluid to a subglottic portion of the trachea via the lower fenestration, and configured to remove fluid therefrom; an upper sealing cuff disposed between the proximal and distal ends of the elongate shaft and superior to the lower sealing cuff, the upper sealing cuff having an expanded configuration and a collapsed configuration, wherein the upper sealing cuff is configured to be disposed in a region above the vocal chords in the patient, and in the expanded configuration the upper sealing cuff is configured to form a seal between the elongate shaft and adjacent tissue; an upper sealing cuff lumen fluidly coupled with the upper sealing cuff and extending proximally toward the proximal end of the elongate shaft, the upper sealing cuff lumen configured to deliver an inflation fluid to and from the upper sealing cuff for inflation and deflation thereof; an upper fenestration in the wall of the elongate shaft, the upper fenestration disposed adjacent the upper sealing cuff and superior thereto; and an upper fenestration lumen fluidly coupled with the upper fenestration and extending proximally therefrom toward the proximal end of the elongate shaft, the upper fenestration lumen configured to deliver fluid to the region above the vocal chords via the upper fenestration and configured to remove fluid therefrom.

Example 2 is the endotracheal tube of Example 1, further comprising a lower sealing cuff inflation port adjacent the proximal end of the elongate shaft, the lower sealing cuff inflation port fluidly coupled with the lower sealing cuff lumen.

Example 3 is the endotracheal tube of any of Examples 1-2, further comprising a lower suction/instillation port adjacent the proximal end of the elongate shaft, the lower port fluidly coupled with the lower fenestration lumen.

Example 4 is the endotracheal tube of any of Examples 1-3, further comprising an upper sealing cuff inflation port adjacent the proximal end of the elongate shaft, the upper sealing cuff inflation port fluidly coupled with the upper sealing cuff lumen.

Example 5 is the endotracheal tube of any of Examples 1-4, further comprising an upper suction/instillation port adjacent the proximal end of the elongate shaft, the upper port fluidly coupled with the upper fenestration lumen.

Example 6 is the endotracheal tube of any of Examples 1-5, further comprising a proximal orogastric fenestration disposed in the wall of the elongate shaft and adjacent the proximal end of the elongate shaft, a distal orogastric fenestration disposed in the wall of the elongate shaft and adjacent the upper sealing cuff and inferior thereto, and an orogastric lumen extending between the proximal and distal orogastric fenestrations, the orogastric lumen configured to slidably receive an orogastric tube.

Example 7 is the endotracheal tube of any of Examples 1-6, wherein the orogastric lumen is disposed in the wall of the elongate shaft.

Example 8 is the endotracheal tube of any of Examples 1-7, further comprising a tube having a lumen extending therethrough, the tube coupled to the proximal orogastric fenestration and configured to slidably receive the orogastric feeding tube.

Example 9 is the endotracheal tube of any of Examples 1-8, wherein the tube comprises a first portion and second portion, and wherein a rigidity of the first portion is different than a rigidity of the second portion.

Example 10 is the endotracheal tube of any of Examples 1-9, further comprising one or more ports, the one or more ports comprising a lower sealing cuff inflation port, lower suction/instillation port, upper sealing cuff inflation port, or an upper suction/instillation port, and wherein the one or more ports are coupled to the endotracheal tube at a position on the endotracheal tube that is intermediate of the proximal end of the endotracheal tube and the upper sealing cuff, the intermediate portion configured to be disposed in an oral cavity of the patient.

Example 11 is the endotracheal tube of any of Examples 1-10, further comprising a mouth cover coupled with the elongate shaft, and configured to form a seal against a patient's mouth.

Example 12 is the endotracheal tube of any of Examples 1-11, wherein the mouth cover comprises a separatable slit configured to receive the endotracheal tube, and wherein the mouth cover further comprises a fastener configured to couple edges of the mouth cover disposed on either side of the slit together.

Example 13 is the endotracheal tube of any of Examples 1-12, wherein the mouth cover comprises a separable orogastric slit configured to receive an orogastric feeding tube, and wherein the mouth cover further comprises a fastener configured to couple edges of the mouth cover disposed on either side of the orogastric slit together.

Example 14 is the endotracheal tube of any of Examples 1-13, further comprising one or more fenestrations disposed in the mouth cover, the one or more fenestrations configured to slidably receive one or more of a lower sealing cuff inflation port, lower suction/instillation port, upper sealing cuff inflation port, or an upper suction/instillation port.

Example 15 is the endotracheal tube of any of Examples 1-14, wherein the mouth cover comprises two or more discrete portions releasably coupled together.

Example 16 is the endotracheal tube of any of Examples 1-15, wherein the mouth cover comprises one or more irrigation ports configured to deliver fluid into the patient's mouth.

Example 17 is the endotracheal tube of any of Examples 1-16, further comprising a sealing element configured to seal the mouth cover with the elongate shaft or to seal the mouth cover with a face of the patient.

Example 18 is the endotracheal tube of any of Examples 1-17, wherein the mouth cover comprises a flange configured to be disposed in a mouth of the patient posterior to teeth in the mouth, or wherein the flange is configured to be disposed between a lip of the patient and the teeth.

Example 19 is the endotracheal tube of any of Examples 1-18, further comprising a fastener element coupled to the mouthpiece, the fastener element configured to be coupled with a headband or a strap that is configured to be at least partially disposed around a head of the patient.

Example 20 is the endotracheal tube of any of Examples 1-19, further comprising the headband or the strap.

Example 21 is the endotracheal tube of any of Examples 1-20, wherein one or more of the lower sealing cuff lumen, the lower fenestration lumen, the upper sealing cuff lumen, and the upper fenestration lumen is disposed in the wall of the elongate shaft.

Example 22 is the endotracheal tube of any of Examples 1-21, wherein the proximal end of the elongate shaft comprises a connector element for releasably coupling with a cooperating connector element for fluidly coupling the tracheal lumen with a ventilator.

Example 23 is an endotracheal tubing system comprising: an endotracheal tube comprising an elongate shaft with a lower sealing cuff configured to form a seal in a larynx of a patient, a lower fenestration superior to the lower sealing cuff and configured to remove fluid from or deliver fluid to a region in the patient, an upper sealing cuff configured to form a seal in a region above the vocal chords of the larynx of the patient, an upper fenestration superior to the upper sealing cuff and configured to remove fluid from or deliver fluid to the region above the vocal chords of the patient; and a ventilator fluidly coupled with the endotracheal tube.

Example 24 is the system of Example 23, further comprising a mouth cover coupled with the endotracheal tube and configured to seal a mouth of the patient.

Example 25 is the system of any of Examples 23-24, wherein the mouth cover comprises one or more irrigation ports configured to deliver fluid into the patient's mouth.

Example 26 is the system of any of Examples 23-25, further comprising a nasogastric or orogastric feeding tube slidably disposed in a lumen of the endotracheal tube.

Example 27 is the system of any of Examples 23-26, further comprising a sealing element configured to seal the mouth cover with the elongate shaft or to seal the mouth cover with a face of the patient.

Example 28 is the system of any of Examples 23-27, wherein the mouth cover comprises a flange configured to be disposed in a mouth of the patient posterior to teeth in the mouth, or wherein the flange is configured to be disposed between a lip of the patient and the teeth.

Example 29 is the system of any of Examples 23-28, further comprising a fastener element coupled the mouth cover, the fastener element configured to be coupled with a headband or a strap that is configured to be at least partially disposed around a head of the patient.

Example 30 is the system of any of Examples 23-29, further comprising the headband or the strap.

Example 31 is a method for intubating a patient, said method comprising: inflating a lower sealing cuff on an endotracheal tube to form a seal between the endotracheal tube and a trachea; instilling a first fluid through a lower lumen in the endotracheal tube and out a lower fenestration in a wall of the endotracheal tube to a first region of the patient; removing the first fluid and secretions from the first region via the lower fenestration and lower fenestration lumen; inflating an upper sealing cuff on the endotracheal tube to form a seal between the endotracheal tube and tissue in a region above the vocal chords of the patient; cleaning lips and a mouth of the patient; covering the mouth with a mouth cover and sealing the mouth; introducing a nasal fluid into one or more nares, wherein the mouth cover prevents the nasal fluid from exiting the mouth; removing the nasal fluid and nasal secretions via an upper lumen disposed in the endotracheal tube and an upper fenestration disposed in the wall of the endotracheal tube, the upper fenestration disposed superior to the upper sealing cuff; removing the mouth cover; instilling an antiseptic into the mouth and removing the antiseptic from the mouth; deflating the upper sealing cuff; and instilling the first fluid into the first region via the lower fenestration lumen and lower fenestration, and removing the first fluid and secretions via the lower fenestration and lower fenestration lumen.

Example 32 is the method of Example 31, further comprising inserting the endotracheal tube into the trachea of the patient.

Example 33 is the method of any of Examples 31-32, wherein the endotracheal tube comprises one or more inflation lines, suction lines, or irrigation lines coupled to the endotracheal tube at a coupling position along the endotracheal tube, and wherein inserting the endotracheal tube comprises advancing the endotracheal tube into the trachea only so far such that the coupling position remains disposed in an oral cavity of the patient.

Example 34 is the method of any of Examples 31-33, further comprising adjusting a position of the patient prior to inserting the endotracheal tube into the trachea.

Example 35 is the method of any of Examples 31-34, further comprising sedating the patient prior to inserting the endotracheal tube into the trachea.

Example 36 is the method of any of Examples 31-35, wherein inflating the lower sealing cuff comprises overinflating the lower sealing cuff.

Example 37 is the method of any of Examples 31-36, wherein the first fluid comprises saline.

Example 38 is the method of any of Examples 31-37, wherein cleaning the lips and the mouth comprises brushing teeth of the patient, cleaning buccal regions, or cleaning a tongue of the patient.

Example 39 is the method of any of Examples 31-38, wherein the nasal fluid comprises saline or an atomized fluid.

Example 40 is the method of any of Examples 31-39, wherein introducing the nasal fluid into one or more nares comprises introducing the nasal fluid into one nostril while occluding an opposite nostril.

Example 41 is the method of any of Examples 31-40, wherein the antiseptic comprises chlorhexidine, an antibiotic, or a lubricant.

Example 42 is the method of any of Examples 31-41, wherein removing the antiseptic comprises suctioning the antiseptic and other secretions out of the mouth with a suction wand.

Example 43 is the method of any of Examples 31-42, further comprising decreasing pressure in the lower sealing cuff after cleaning the mouth and the nares while still providing an adequate seal between the lower sealing cuff and the trachea.

Example 44 is the method of any of Examples 31-43, wherein after patient intubation and stabilization, oral and nasal irrigation commences, and nasal and oral cleaning is complete in 5 minutes or less.

Example 45 is the method of any of Examples 31-44, further comprising preventing or minimizing aerosolized fluids from exiting a mouth of the patient.

Example 46 is the method of any of Examples 31-45, wherein instilling the antiseptic into the mouth comprises introducing the antiseptic into the mouth via one or more irrigation ports on the mouth cover.

Example 47 is the method of any of Examples 31-46, further comprising sealing the mouth cover with the elongate shaft with a sealing element, or sealing the mouth cover with a face of the patient with the sealing element.

Example 48 is the method of any of Examples 31-47, wherein the mouth cover comprises a flange, the method further comprising disposing the flange in a mouth of the patient posterior to teeth in the mouth, or disposing the flange between a lip of the patient and the teeth.

Example 49 is the method of any of Examples 31-48, wherein covering the mouth with the mouth cover comprises enlarging a slit region on the mouth cover and sliding the enlarged slit region over the endotracheal tube.

Example 50 is the method of any of Examples 31-49, further comprising closing the enlarged slit region and fastening edges along the slit region together with a fastener.

Example 51 is the method of any of Examples 31-50, wherein covering the mouth with the mouth cover comprises enlarging a slit region on the mouth cover and sliding the enlarged slit region over an orogastric feeding tube.

Example 52 is the method of any of Examples 31-51, further comprising closing the enlarged slit region and fastening edges along the slit region together with a fastener.

Example 53 is the method of any of Examples 31-52, wherein the endotracheal tube further comprises one or more of a lower sealing cuff inflation port, a lower suction/installation port, an upper sealing cuff port, or an upper suction/instillation port, and wherein covering the mouth with the mouth cover comprises passing one or more of the lower sealing cuff inflation port, the lower suction/installation port, the upper sealing cuff port, or the upper suction/instillation ports through an aperture in the mouth cover.

Example 54 is the method of any of Examples 31-53, wherein the mouth cover comprises a plurality of pads, and wherein covering the mouth with the mouth cover comprises surrounding the endotracheal tube with the plurality of pads and joining the plurality of pads together with a fastener.

Example 55 is the method of any of Examples 31-54, further comprising disposing a headband or a strap at least partially around a head of the patient and coupling the headband or the strap to the mouth cover.

Example 56 is the method of any of Examples 31-55, further comprising slidably advancing an orogastric feeding tube through the endotracheal tube.

Example 57 is an endotracheal tube with a pharyngeal fluid line configured to irrigate a region above a patient's vocal chords with high pressure fluid or fluid at a high flow rate to dislodge and remove organic debris and secretions therefrom.

Example 58 is a system to reduce VAP configured to introduce lavage fluid to a nasal and an oral cavity with no dwell time by simultaneously applying high pressure suctioning to a region above the vocal chords while avoiding aspiration of the fluid into a lung of a patient.

Example 59 is an endotracheal tube with a pharyngeal suction line configured to remove organic debris and thick secretions from a region above a vocal chord of a patient including a nasal cavity or an oral cavity using high suction.

Example 60 is an endotracheal tube that comprises a closed circuit for introducing fluid and suctioning the fluid from a nasopharyngeal region, wherein the fluid is introduced via a nasal atomizer, and the closed circuit is partially formed with a mouth pad disposed against a patient's mouth, and wherein the endotracheal tube comprises a suction fenestration disposed in the nasopharyngeal region.

Example 61 is a system comprising an endotracheal tube configured to remove endogenous bacteria from nasal and oral cavities, wherein the endotracheal tube comprises an expandable cuff configured to be disposed in a region above a vocal chord, a lower fenestration adjacent and proximal to the expandable cuff, and a suction lumen integral with the endotracheal tube and fluidly coupled with the lower fenestration, wherein the lower fenestration is disposed on a posterior side of the endotracheal tube, and wherein the expandable cuff is configured to occlude a pharynx thereby preventing aspiration of fluids or debris into a lung of patient.

Example 62 is an endotracheal tube having a lumen integral with the endotracheal tube, and having walls formed of a material that is also used to form the endotracheal tube, the lumen configured to slidably receive an orogastric feeding tube, and wherein the orogastric feeding tube exits the lumen adjacent a first expandable cuff, the first expandable cuff disposed a distance away from a second expandable cuff on the endotracheal tube, the distance being 30 to 45% of an overall length of the endotracheal tube.

Example 63 is an endotracheal tube configured to separate a patient's nasal and oral cavities from a lower respiratory tract such that oral and nasal irrigant is not aspirated into a lung of the patient.

Example 64 is a method for lavage, the method comprising introducing lavage fluid through nares, a mouth, or an oropharynx or hypopharynx region at flow rates of 3.5 mL per second or greater.

Example 65 is a method of suctioning fluid or dislodged organic material from a patient and removing the fluid or the organic material with a suction strength of up to 150 mm Hg.

Example 66 is an endotracheal tube comprising an oropharyngeal or hypopharyngeal cuff having an expanded configuration and a collapsed configuration, wherein in the collapsed configuration the cuff is biased to collapse toward a posterior portion of the endotracheal tube so that an anterior portion of the endotracheal tube is largely free of the collapsed cuff.

Example 67 is a mouth pad for sealing an intubated patient's mouth, the mouth pad comprising a center aperture configured to receive an endotracheal tube, wherein the center aperture is bounded by semi-rigid material, and wherein a surface of the mouth pad configured to appose the mouth comprises a flexible material with an adhesive disposed thereon.

Example 68 is an endotracheal tube comprising a pharyngeal cuff configuration to expand and form a seal that prevents secretions from crossing the cuff and moving distally down the endotracheal tube.

Example 69 is an endotracheal tube having an expandable cuff configured to expand and form a seal that blocks secretions from crossing the cuff, the endotracheal tube further comprising means for suctioning large volumes of secretions and bleeding.

Example 70 is an endotracheal tube system, comprising: an endotracheal tube; a mouth pad having a central aperture, wherein the endotracheal tube is disposed in the central aperture; an irrigation line having a plurality of irrigation fenestrations coupled to the mouth pad; a suction line having a plurality of suction fenestrations coupled to the mouth pad; and a line protector coupled to the mouth pad, wherein the line protector is disposed around the irrigation line and the suction line to provide protection thereto.

Example 71 is the endotracheal tube system of Example 70, further comprising a flanged region coupled to the line protector, the flanged region configured to be placed behind a patient's upper and lower front teeth.

Example 72 is a device for irrigation of an oral cavity in a patient, the device comprising: a suction element coupled to the irrigation plate, the suction element configured to be disposed in the oral cavity and configured to suction a the fluid out of the oral cavity, the suction element further comprising one or more irrigation outlets configured to irrigate the oral cavity with the fluid; and a suction line fluidly coupled with the suction element, the suction line configured to be fluidly coupled with a vacuum source; and an irrigation line fluidly coupled with the one or more irrigation outlets, the irrigation line fluidly coupled with a source of the fluid.

Example 73 is the device of Example 72, further comprising an irrigation plate comprising one or more irrigation outlets configured to irrigate the oral cavity with the fluid, the irrigation plate coupled with the suction element.

Example 74 is the device of any of Examples 72-73, wherein the irrigation plate comprises a flat planar disc.

Example 75 is the device of any of Examples 72-74, wherein the irrigation plate comprises a reservoir configured to hold the fluid.

Example 76 is the device of any of Examples 72-75, further comprising a teeth registration element coupled to the irrigation plate or the suction element, the teeth registration element configured to facilitate alignment of the device with teeth in a mouth of the patient.

Example 77 is the device of any of Examples 72-76, wherein the teeth registration element comprises anterior and posterior walls coupled to the irrigation plate or the suction element and extending downward therefrom, the anterior and posterior walls forming a gap therebetween, the gap configured to receive lower teeth of the patient thereby preventing or minimizing anterior or posterior movement of the irrigation plate in the oral cavity.

Example 78 is the device of any of Examples 72-77, wherein the one or more irrigation outlets comprise a plurality of irrigation outlets disposed on an upper or lower surface of the irrigation plate.

Example 79 is the device of any of Examples 72-78, wherein the one or more irrigation outlets comprise a plurality of irrigation outlets disposed around a perimeter of the irrigation plate.

Example 80 is the device of any of Examples 72-79, wherein the irrigation line comprises connector element coupled to a proximal end thereof.

Example 81 is the device of any of Examples 72-80, wherein the irrigation plate comprises a first edge, a second edge opposite the first edge and an intermediate portion disposed therebetween, and wherein the cradle comprises a plurality of protrusions extending away from an upper surface of the irrigation plate and separated from one another by a gap in the intermediate portion of the irrigation plate, wherein the gap is sized and shaped to receive the endotracheal tube and the protrusions are configured to engage and hold the endotracheal tube.

Example 82 is the device of any of Examples 72-81, wherein the suction element comprises one or more paddles, wherein each paddle comprises an elongated arm and an enlarged head.

Example 83 is the device of any of Examples 72-82, wherein the one or more paddles comprise a plurality of paddles coupled together to form a U-shape or a V-shape.

Example 84 is the device of any of Examples 72-83, wherein the enlarged head is configured to be disposed in a buccal pocket between a cheek and teeth of the patient, and wherein when the patient is disposed on a lateral side, the fluid accumulates in the buccal pocket and is suctioned therefrom.

Example 85 is the device of any of Examples 72-84, wherein the enlarged head comprises a disc with one or more suction inlets disposed thereon, the enlarged head configured to be disposed between a cheek and a teeth in the patient.

Example 86 is the device of any of Examples 72-85, wherein the elongated arm or the enlarged head comprises one or suction inlets or one or more irrigation outlets disposed thereon.

Example 87 is the device of any of Examples 72-86, wherein the suction element comprises one or more suction inlets disposed thereon, and wherein the suction element further comprises a suction guard adjacent the one or more suction inlets, the suction guard configured to prevent or minimize tissue from being drawn into the one or more suction inlets.

Example 88 is the device of any of Examples 72-87, wherein the suction line comprises a plurality of suction lines fluidly coupled to the suction element.

Example 89 is the device of any of Examples 72-88, wherein the plurality of suction lines comprise a connector coupled to a proximal end thereof.

Example 90 is a system for irrigation of an oral cavity in a patient, the system comprising the device of any of Examples 72-89; and an endotracheal tube releasably coupled to the device.

Example 91 is the system of Example 90, further comprising the fluid.

Example 92 is the system of any of Examples 90-91, further comprising a mouth pad coupled to the endotracheal tube, the mouth pad configured to prevent fluids, droplets or aerosols from escaping the oral cavity.

Example 93 is a method for irrigating an oral cavity in a patient, the method comprising: coupling an oral cavity irrigation device to an endotracheal tube; irrigating the oral cavity with a fluid delivered by the oral cavity irrigation device; and suctioning the fluid out of the oral cavity with the oral cavity irrigation device.

Example 94 is the method of Example 93, wherein the coupling occurs after the endotracheal tube is disposed in a patient's trachea.

Example 95 is the method of any of Examples 93-94, wherein the fluid is an antiseptic, a lubricant, water, saline, or an antibiotic.

Example 96 is the method of any of Examples 93-95, wherein irrigating the oral cavity comprises irrigating an upper portion of the oral cavity or a lower portion of the oral cavity with the oral cavity irrigation device.

Example 97 is the method of any of Examples 93-96, wherein the suctioning is performed while patient is disposed on a lateral side to form a low point in a buccal pocket of the oral cavity where the fluid accumulates and is suctioned therefrom.

Example 98 is the method of any of Examples 93-97, wherein the oral cavity irrigation device comprises a suctioning element, and wherein the suctioning is performed by the suctioning element, and wherein the suctioning element is disposed between a cheek of the patient and adjacent teeth of the patient.

Example 99 is the method of any of Examples 93-98, wherein the coupling comprises inserting the endotracheal tube into a cradle coupled to the oral irrigation device.

Example 100 is the method of any of Examples 93-99, further comprising coupling a mouth pad to the endotracheal tube and sealing the mouth pad against a mouth of the patient, thereby preventing fluids from escaping from the mouth.

Example 101 is a method of treating an oral cavity in a patient, the method comprising: coupling an irrigation device to an endotracheal tube disposed in the oral cavity of a ventilated patient; positioning the patient on a side so a buccal pocket in the oral cavity is disposed in a low position relative to other regions of the oral cavity; irrigating the oral cavity with a fluid from the irrigation device; and suctioning the fluid out of the buccal pocket.

In Example 102, the apparatuses, systems, or methods of any one or any combination of Examples 1-101 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1-70. (canceled)

71. A device for irrigation of an oral cavity in a patient, the device comprising:
a suction element configured to be disposed in the oral cavity and configured to suction a fluid out of the oral cavity, the suction element further comprising a first set of one or more irrigation outlets configured to irrigate the oral cavity with the fluid;
a suction line fluidly coupled with the suction element, the suction line configured to be fluidly coupled with a vacuum source;
an irrigation line fluidly coupled with the one or more irrigation outlets, the irrigation line fluidly coupled with a source of the fluid; and
an irrigation plate comprising a second set of one or more irrigation outlets configured to irrigate the oral cavity with the fluid, the irrigation plate comprising a flat planar disc and coupled with the suction element, wherein the irrigation plate is configured to overlie a tongue within the oral cavity and is spaced apart from a cheek of the patient.

72. The device of claim 71, wherein the irrigation plate comprises a reservoir configured to hold the fluid.

73. The device of claim 71, further comprising a teeth registration element coupled to the irrigation plate or the suction element, the teeth registration element configured to facilitate alignment of the device with teeth in a mouth of the patient.

74. The device of claim 73, wherein the teeth registration element comprises anterior and posterior walls coupled to the irrigation plate or the suction element and extending downward therefrom, the anterior and posterior walls forming a gap therebetween, the gap configured to receive lower teeth of the patient thereby preventing or minimizing anterior or posterior movement of the irrigation plate in the oral cavity.

75. The device of claim 71, wherein the one or more irrigation outlets comprise a plurality of irrigation outlets disposed on an upper or lower surface of the irrigation plate.

76. The device of claim 71, wherein the one or more irrigation outlets comprise a plurality of irrigation outlets disposed around a perimeter of the irrigation plate.

77. (canceled)

78. The device of claim 71, wherein the irrigation plate comprises a first edge, a second edge opposite the first edge and an intermediate portion disposed therebetween, and
wherein the cradle comprises a plurality of protrusions extending away from an upper surface of the irrigation plate and separated from one another by a gap in the intermediate portion of the irrigation plate, wherein the gap is sized and shaped to receive the endotracheal tube and the protrusions are configured to engage and hold the endotracheal tube.

79. The device of claim 71, wherein the suction element comprises one or more paddles, wherein each paddle comprises an elongated arm and an enlarged head.

80. (canceled)

81. The device of claim 79, wherein the enlarged head is configured to be disposed in a buccal pocket between a cheek and teeth of the patient, and wherein when the patient is disposed on a lateral side, the fluid accumulates in the buccal pocket and is suctioned therefrom.

82. The device of claim 79, wherein the enlarged head comprises a disc with one or more suction inlets disposed thereon, the enlarged head configured to be disposed between a cheek and teeth in the patient.

83. The device of claim 79, wherein the elongated arm or the enlarged head comprises one or more suction inlets or one or more irrigation outlets disposed thereon.

84. The device of claim 71, wherein the suction element comprises one or more suction inlets disposed thereon, and wherein the suction element further comprises a suction guard adjacent the one or more suction inlets, the suction guard configured to prevent or minimize tissue from being drawn into the one or more suction inlets.

85. The device of claim 71, wherein the suction line comprises a plurality of suction lines fluidly coupled to the suction element, the plurality of suction lines comprise a connector coupled to a proximal end thereof and the irrigation line comprises a connector element coupled to a proximal end thereof.

86-89. (canceled)

90. A method for irrigating an oral cavity in a patient, the method comprising:
coupling the oral cavity irrigation device of claim 71 and an endotracheal tube that are together disposed in the oral cavity of the patient;
irrigating the oral cavity with a fluid delivered by the oral cavity irrigation device; and
suctioning the fluid out of the oral cavity with the oral cavity irrigation device.

91. (canceled)

92. The method of claim 90, wherein the fluid is an antiseptic, a lubricant, water, saline, or an antibiotic.

93. The method of claim 90, wherein irrigating the oral cavity comprises irrigating an upper portion of the oral cavity or a lower portion of the oral cavity with the oral cavity irrigation device.

94. The method of claim 90, wherein the suctioning is performed while patient is disposed on a lateral side to form a low point in a buccal pocket of the oral cavity where the fluid accumulates and is suctioned therefrom.

95. The method of claim 90, wherein the oral cavity irrigation device comprises a suctioning element, and wherein the suctioning is performed by the suctioning element, and wherein the suctioning element is disposed between a cheek of the patient and adjacent teeth of the patient.

96. The method of claim 90, wherein the coupling comprises inserting the endotracheal tube into a cradle coupled to the oral irrigation device.

97. The method of claim 90, further comprising coupling a mouth pad to the endotracheal tube and sealing the mouth pad against a mouth of the patient, thereby preventing fluids from escaping the mouth.

98. (canceled)

99. A medical device for suction and irrigation of an oral cavity in a patient, wherein the device comprises:
a suction element configured to be placed within the oral cavity and comprising:
a first paddle for placement adjacent upper and lower teeth at one side of the oral cavity of the patient, the first paddle further comprising at least one suction inlet configured to suction fluid out of the oral cavity; and
a second paddle for placement adjacent the upper and lower teeth at another side of the oral cavity of the patient, the second paddle further comprising one or more irrigation outlets configured to irrigate the oral cavity with fluid;
a suction port that is fluidically coupled to the first paddle for providing a negative pressure thereto for suctioning the oral cavity during use; and
an irrigation port that is fluidically coupled to the second paddle for providing the fluid thereto for irrigating the oral cavity during use.

100. The medical device of claim 99, wherein the first paddle comprises an elongated arm and an enlarged head.

101. The medical device of claim 99, wherein the second paddle comprises an elongated arm having an elliptical shape.

102. The medical device of claim 99, wherein a suction line is fluidly coupled to the suction port.

103. The medical device of claim 99, wherein an irrigation line is fluidly coupled to the irrigation port.
